(19) United States
(12) Patent Application Publication
(10) Pub. No.: US 2025/0352268 A1
(43) Pub. Date: Nov. 20, 2025

(54) METHOD AND APPARATUS FOR PLANNING PLACEMENT OF AN IMPLANT

(71) Applicant: Medtronic, Inc., Minneapolis, MN (US)

(72) Inventors: Frans L.H. GIELEN, Eckelrade (NL); Juergen Konrad MAI, Düsseldorf (DE); Milan MAJTANIK, Viersen (DE)

(21) Appl. No.: 18/869,713

(22) PCT Filed: May 23, 2023
(86) PCT No.: PCT/US2023/023190

§ 371 Date: Nov. 26, 2024

Related U.S. Application Data
(60) Provisional application No. 63/346,373, filed on May 27, 2022.
(60) Provisional application No. 63/346,380, filed on May 27, 2022.
(60) Provisional application No. 63/346,393, filed on May 27, 2022.
(60) Provisional application No. 63/346,400, filed on May 27, 2022.

(51) Int. Cl. A61B 34/10; A61N 1/05; G06N 3/08

(52) U.S. Cl. CPC A61B 34/10; G06N 3/08; A61B 2034/104; A61B 2034/105; A61N 1/0534

(57) ABSTRACT

Disclosed is a system to plan and position an implant in a subject. The planned position may be based upon various features and structures identified in a group of subjects for a current subject. The implant may then be positioned in a selected position which may be identified as an optimal position for the selected current subject.

### CROSS-REFERENCE TO RELATED APPLICATIONS

This application claims the benefit of U.S. Provisional App. No. 63/346,373, filed May 27, 2022 (5074N-000040-US-PS1), U.S. Provisional App. No. 63/346,380, filed May 27, 2022 (5074N-000041-US-PS1), U.S. Provisional App. No. 63/346,393, filed May 27, 2022 (5074N-000042-US-PS1), and U.S. Provisional App. No. 63/346,400, filed May 27, 2022 (5074N-000043-US-PS1). The entire disclosures of each of the above applications are incorporated herein by reference.

### FIELD

The present disclosure is related to a system for planning a procedure on a subject, in particularly, to evaluating features of a subject to select placement of an implant in the subject.

### BACKGROUND

This section provides background information related to the present disclosure which is not necessarily prior art.

In performing a procedure on a subject, such as a human subject, implants may be positioned in the human subject for various purposes. For example, an implant may be positioned within a brain of a human to provide stimulation or therapy at selected positions therein. Therapy within the brain, however, may vary in efficacy, speed, and the like based upon various parameters of the implantation including implantation location, brain network specifics, delivery features, or the like.

According to various systems, an implant may be positioned within a subject to provide therapy thereto. The therapy may include electrical stimulation of portions of the brain adjacent to the implant. In various embodiments, for example, diffusion tensor image data may be used during a selected portion of a procedure, as disclosed in U.S. Pat. No. 8,532,741. In various embodiments, a non-electrical stimulation may also be provided through an implant or an instrument, such as the delivery of a pharmaceutical agent. The pharmaceutical agent may be delivered with a selected device and various image data may be analyzed to assist in determining a placement or delivery location for the pharmaceutical, such as disclosed at U.S. Pat. No. 8,335,552.

### SUMMARY

This section provides a general summary of the disclosure, and is not a comprehensive disclosure of its full scope or all of its features.

A portion of a human anatomy can be imaged to generate image data thereof that may be analyzed as data and/or visually by a user. In analyzing the image data, various features may be identified and/or used to identify various additional features. For example, an anatomical landmark may be identifiable in a selected image data and may be used to assist in identifying a region or portion of image data that is not visually distinguishable therefrom. Various types of image data may include magnetic resonance imaging (MRI) data, x-ray image data, functional MRI data, diffusion data (such as MRI DTI data), brain atlas data, anatomical data, and other appropriate information. According to various embodiments, for example, image data may be acquired of a subject and landmarks, such as brain network connections and/or anatomical landmarks, may be identified therein and other anatomical features identified relative thereto. For example, an anterior commissure and a posterior commissure may be identified in image data to assist in locating and/or approximating locations of a sub-thalamic nucleus in the image based upon a pre-determined co-location of the sub-thalamic nucleus relative to the anterior commissure and the posterior commissure such as disclosed at U.S. Pat. No. 8,160,677, incorporated herein by reference. Without being bound by the theory, various landmarks and/or portions of the brain may be understood as one or a set of brain network connections, connecting the brain anatomical structures. Brain anatomy may understood to be a local aspect of the brain, while brain network connections can connect brain anatomy remotely from each other in the brain.

In various embodiments, an implant may be positioned relative to an identified feature in the image, such as a deep-brain stimulation (DBS) device. The DBS may also be referred to as and/or understood as a neuromodulation device and may provide neuromodulation of an appropriate type, such as an electrical current and/or voltage. The therapy provided to the brain may be a neuromodulation. The neuromodulation may include a stimulation to the brain, as discussed herein, may be a stimulation including an electrical stimulation, such as an application of a voltage, amperage, etc. As used herein, stimulation therefore, including electrical stimulation, may refer to neuromodulation. Thus, neuromodulation which may be application of an electrical feature (e.g., amperage, voltage, etc.) may be referred to as a stimulation and may result in either or both of an excitatory or inhibitory response in a brain network.

Further, discussion of a voltage herein may be understood to refer to an amperage (e.g., milliamps (mA)), a potential, or other appropriate electrical application to the subject. The treatment device, such as the DBS, may be used to provide therapy to a subject. In various embodiments, features identified in the image data may include an anterior nucleus of the thalamus (ANT) also referred to as the anterior thalamic nucleus (ATN). Stimulation of the ANT and/or portions of the brain relative to the ANT may assist in therapy for patients diagnosed with various conditions, e.g., epilepsy. In particular, a treatment, such as a stimulation, may assist in epilepsy treatment or therapy for patients that are pharmaco-resistant. Accordingly, analysis of image data may assist in identifying the ANT and/or portions of the brain relative to the ANT. These portions may be used for various purposes, such as therapy for epilepsy.

In various embodiments, a method and/or system may be useful in identifying the ANT and/or locations for positioning an implant in and/or relative to the ANT for providing a therapy. Image data may be analyzed to determine possible locations, optimal locations, or locations selected for achieving selected therapy results in a patient. In addition to the identification of the location of the ANT, data regarding types of therapy, results of therapy, timing of therapy, and parameters of therapy may be used to identify selected locations for positioning an implant and/or parameters of stimulation to achieve a selected result within the subject. Accordingly, the system may be used to identify implant positions and/or parameters for therapy.

The system may also use selected processes, such as machine learning or an application of machine learning, to identify positions of an implant for achieving selected results. The position of the implant may include a position within the subject including a three-dimensional position within the subject relative to various portions of the brain and/or absolute positions based upon various data, such as image data. In addition to an/or alternatively to various physical locations relative to an anatomy, various brain networks may be considered. For instance, Brain networks are crucial if a particular type of epilepsy does not originate in the ANT. Analysis and/or consideration of brain networks may allow electrical stimulation in the ANT area to interact with the area where the seizure originates. Brain Network connections may be vital to achieve this interaction.

The parameters may also include provision of therapy pulses, therapy over multiple electrodes, shape of stimulation, electrode positioning, or the like. Accordingly, the system may allow for planning the position of an implant, parameters for stimulation to provide therapy, and proposed outcome timing and results based upon the planned position and parameters.

In various embodiments, a method of evaluating a current subject data to plan a position for placement of an implant in a current subject is disclosed. The method may include at least one or more of accessing an optimality space including at least one feature space regarding a specific therapy and at least one structure space regarding a specific therapy, wherein the feature space and the structure space includes data regarding possible positions of the implant, possible therapies with the implant, and possible outcomes related to the possible positions and therapies; accessing a current subject data regarding the specific therapy; warping the optimality space to the current subject data; receiving a feature space weight for the feature space and a structure space weight for the structure space; evaluating the accessed optimality space and the received feature space weight and structure space weight; determining at least a target for the implant based on the evaluation; and outputting a position of the target in the current subject data.

In various embodiments, a system operable to evaluate a current subject data to plan a position for placement of an implant in a current subject is disclosed. The system may include at least one or more of a processor module configured to execute instructions. The instructions may include at least one or more of access an optimality space from a memory including at least one feature space regarding a specific therapy and at least one structure space regarding a specific therapy, wherein the feature space and the structure space includes data regarding possible positions of the implant, possible therapies with the implant, and possible outcomes related to the possible positions and therapies; access a current subject data regarding the specific therapy;
warp the optimality space to the current subject data; receive a feature space weight for the feature space and a structure space weight for the structure space; determining at least a target for the implant based on an evaluation of the accessed optimality space and the received feature space weight and structure space weight; and output a position of the target in the current subject. The system may include an output system to provide the output position of the target in the current subject data to a user.

In various embodiments, a method of evaluating a current subject data to plan a position for placement of an implant in a current subject is disclosed. The method may include at least one or more of accessing an optimality space including at least one feature space regarding a specific therapy and at least one structure space regarding a specific therapy, wherein the feature space and the structure space includes data regarding possible positions of the implant, possible therapies with the implant, and possible outcomes related to the possible positions and therapies; accessing a current subject data regarding the specific therapy; warping the optimality space to the current subject data; receiving a feature space weight for the feature space and a structure space weight for the structure space; evaluating the accessed optimality space and the received feature space weight and structure space weight; determining at least a target for the implant based on the evaluation; and outputting a position of the target in the current subject data.

In various embodiments, a system operable to evaluate a current subject data to plan a position for placement of an implant in a current subject is disclosed. The system may include a processor module configured to execute instructions. The instructions may include at least one or more of access an optimality space from a memory including at least one feature space regarding a specific therapy and at least one structure space regarding a specific therapy, wherein the feature space and the structure space includes data regarding possible positions of the implant, possible therapies with the implant, and possible outcomes related to the possible positions and therapies; access a current subject data regarding the specific therapy; warp the optimality space to the current subject data; receive a feature space weight for the feature space and a structure space weight for the structure space; determine at least a target for the implant based on an evaluation of the accessed optimality space and the received feature space weight and structure space weight; and output a position of the target in the current subject. The system may further include an output system to provide the output position of the target in the current subject data to a user.

In various embodiments, a method of evaluating a current subject data to plan a placement of an implant in a current subject is disclosed. The method may include at least one or more of accessing an optimality space including a selected plurality of (i) feature spaces, (ii) structure spaces, or (iii) both feature spaces and structure spaces regarding a specific therapy; accessing a current subject data regarding the specific therapy; warping the accessed optimality space to the current subject data; determining a sweet spot target for the implant based on varying a selected weight for each one of the selected feature spaces and structure spaces; and outputting the position of the sweet spot target in the current subject data.

In various embodiments, a system operable to plan a position of an implant in a current subject is disclosed. The system may include at least one or more of a memory system having stored thereon data define an optimality space including a selected plurality of (i) feature spaces, (ii) structure spaces, or (iii) both feature spaces and structure spaces regarding a specific therapy and a processor system configured to execute instructions. The instructions may include one or more of access a current subject data regarding the specific therapy, recall from the memory system the define optimality space, warp the accessed optimality space to the current subject data, determine a sweet spot target for the implant based on varying a selected weight for each one of the selected feature spaces and structure spaces. The system may further include an output system to operable to provide the determined sweet spot to a user.

In various embodiments, a method of generating an optimality space for planning a procedure with a current subject data to plan a position for placement of an implant in a current subject is disclosed. The method may include at least one or more of accessing an optimality space including at least one feature space regarding a specific therapy and at least one structure space regarding a specific therapy, wherein the feature space and the structure space includes data regarding possible positions of the implant, possible therapies with the implant, and possible outcomes related to the possible positions and therapies; generating predictors based on the accessed optimality space; evaluating a validation subject data to predict an outcome based on the generated predictors; determining a similarity between the predicted outcome and a real outcome; if the similarity is below a selected threshold, update the accessed optimality space to generate an updated optimality space; and saving the updated optimality space when generated.

In various embodiments, a system operable to generate an optimality space for planning a procedure with a current subject data to plan a position for placement of an implant in a current subject is disclosed. The system may include at least one or more of a processor module configured to execute instructions. The instructions may include at least one or more of access an optimality space including at least one feature space regarding a specific therapy and at least one structure space regarding a specific therapy, wherein the feature space and the structure space includes data regarding possible positions of the implant, possible therapies with the implant, and possible outcomes related to the possible positions and therapies; determine predictors based on the accessed optimality space; evaluate a validation subject data to predict an outcome based on the generated predictors; determine a similarity between the predicted outcome and a real outcome; determine if the similarity is below a selected threshold to update the accessed optimality space to generate an updated optimality space. The system may further include an output system to output the updated optimality space when generated for recall.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations, and are not intended to limit the scope of the present disclosure.

FIG. 1A is a schematic illustration of a subject and implant device;

FIG. 1B is a schematic cross-sectional view through a brain of the subject of FIG. 1A;

FIG. 2 is a flow chart of a process for selecting a therapy for a particular subject;

FIG. 3 is an illustration of a display of a selected feature of the process of FIG. 2;

FIG. 4 is a graphical representation of a feature of the process of FIG. 2;

FIG. 5 is a graphing of the feature of the process of FIG. 2;

FIGS. 6A-6C are graphical representations of a feature of the process of FIG. 2;

FIG. 7 is a schematic illustration of a feature of the process of FIG. 2;

FIG. 8 is a graphical representation of a feature of the process of FIG. 2;

FIG. 9 is a graphical representation of a feature of the process of FIG. 2;

FIG. 10 is a graphical representation of a feature of the process of FIG. 2;

FIG. 11 is a graphical representation of an evaluation of a feature of FIG. 2;

FIG. 12A is a graphical representation of a selected structure of the process of FIG. 2;

FIG. 12B is a graphical representation of a structure for the process of FIG. 2;

FIG. 12C is a graphical representation of a structure for the process of FIG. 2;

FIG. 13 is a schematic illustration of an application of the process of FIG. 2 to a selected patient;

FIG. 14 is an environmental view of an operating suite;

FIG. 15 is a graphical representation of an outcome for the features and structures of the process of FIG. 2;

FIG. 16 is a graphical representation of the use of the process of FIG. 2; and

FIG. 17 is a flow chart of a process for updating a input of the process of FIG. 2.

Corresponding reference numerals indicate corresponding parts throughout the several views of the drawings.

### DETAILED DESCRIPTION

Example embodiments will now be described more fully with reference to the accompanying drawings.

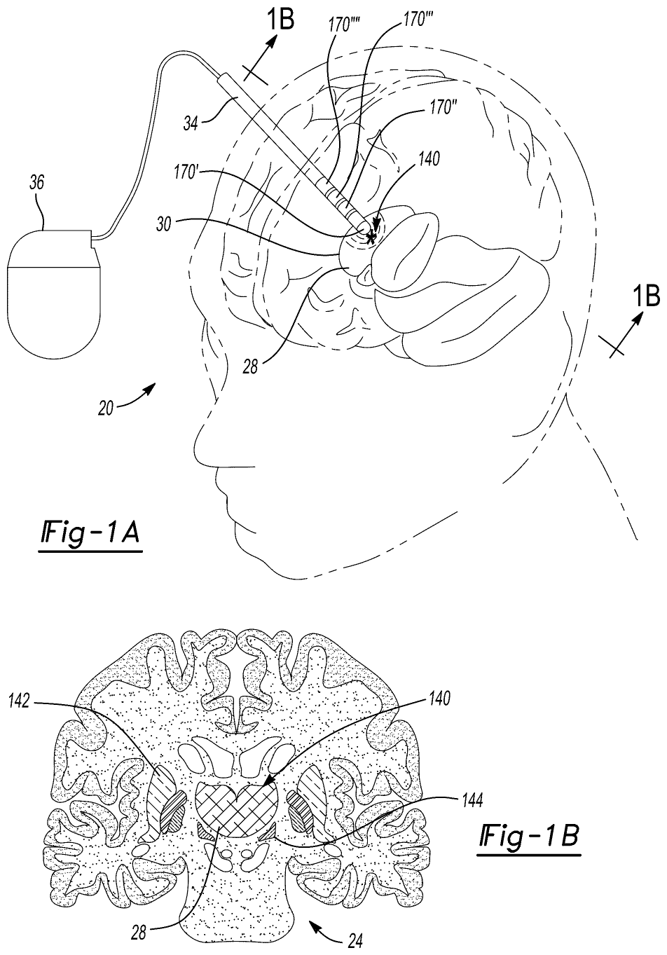

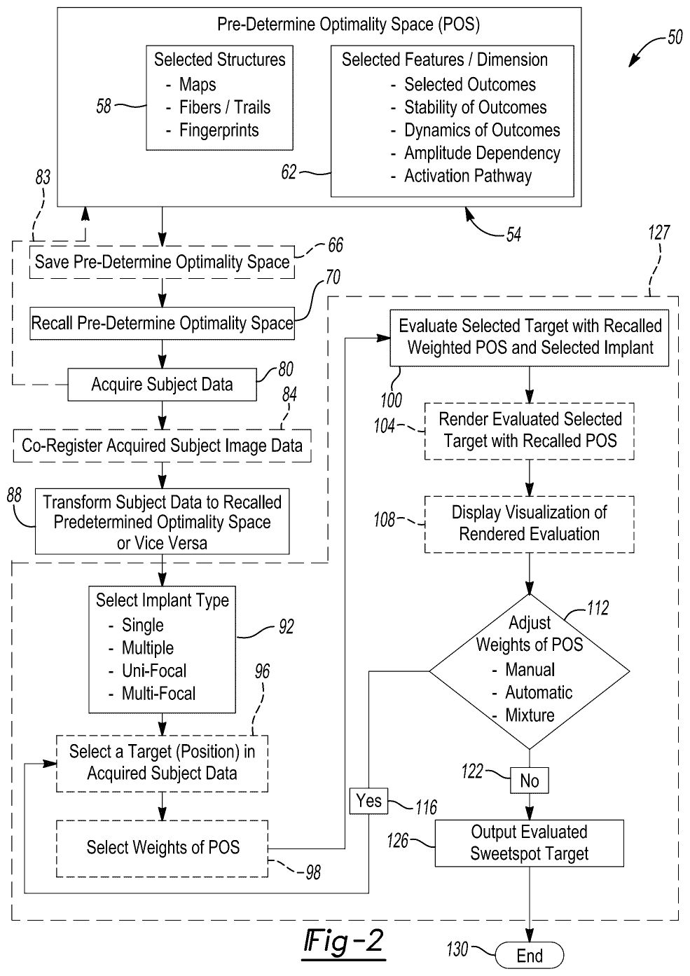

With initial reference to FIG. 1A and FIG. 1B, a subject 20 may be any appropriate subject. Although the following discussion relates to a human subject, it is understood that any appropriate living or non-living subject may be provided or be within the scope of the subject disclosure. For example, a non-human living subject may be evaluated and a selected procedure performed thereon. Further, various non-living subjects may have image data acquired of internal portions and a procedure may be determined, planned, and performed within an outer housing or body (such as a hull) of the non-living subject. Various non-living subjects include internal portions of motors, hulls, or other appropriate subjects. Also, while the following discussion refers exemplarily to placing a deep brain stimulation (DBS) device as an implant for stimulation of a brain, other appropriate implants and/or therapies are within the scope of the subject disclosure.

As noted above, for example, a human subject (also referred to herein as subject) may have a select treatment prescribed therefor. The treatment may include providing various implants into the subject 20, such as into a brain 24 thereof to assist in providing a therapy to the subject. In various embodiments, for example, the subject 20 may be diagnosed with epilepsy and stimulation may be selected for treating or preventing a therapy to the subject 20. It is understood, that the subject 20 may be diagnosed with other disorders and appropriate treatment, according to the subject disclosure, may be determined and/or provided.

In various embodiments, a thalamus 28 may be identified and/or a selected portion thereof, such as an interior portion (e.g., anterior nucleus of the thalamus (ANT) also referred to as an anterior thalamic nucleus (ATN)) 30 may be identified for treatment with a selected therapy. In various embodiments, an implant 34 may be positioned relative to the ANT 30 (i.e., in and/or spaced apart from the ANT 30) for providing therapy to the subject 20. The implant may be positioned at any appropriate position and provide neuromodulation to any selected portion of the brain, such as the ANT 30. Thus, the implant need not be placed in or only in the ANT 30. The implant 34 may be any appropriate implant and may include a deep-brain stimulation probe or implant. Exemplary implants include a SENSIGHT® DBS lead, sold by Medtronic, Inc. having a place of business in Minnesota. It is understood, however, that other appropriate DBS leads may be used for positioning within the subject 20 and a SENSIGHT® DBS lead is merely exemplary. Further, the lead may be connected to a selected stimulator, such as a Percept™ neurostimulator to provide a stimulation therapy to the subject 20. Again, the Percept™ is merely exemplary of any appropriate device or neurostimulator. The stimulator 36 may be programmed in an appropriate manner, such as discussed further herein, to provide a selected therapy to the subject 20. The programming of the stimulator 36 may be based upon various selections and/or determinations, as also discussed further herein, to provide a selected therapy to the subject 20.

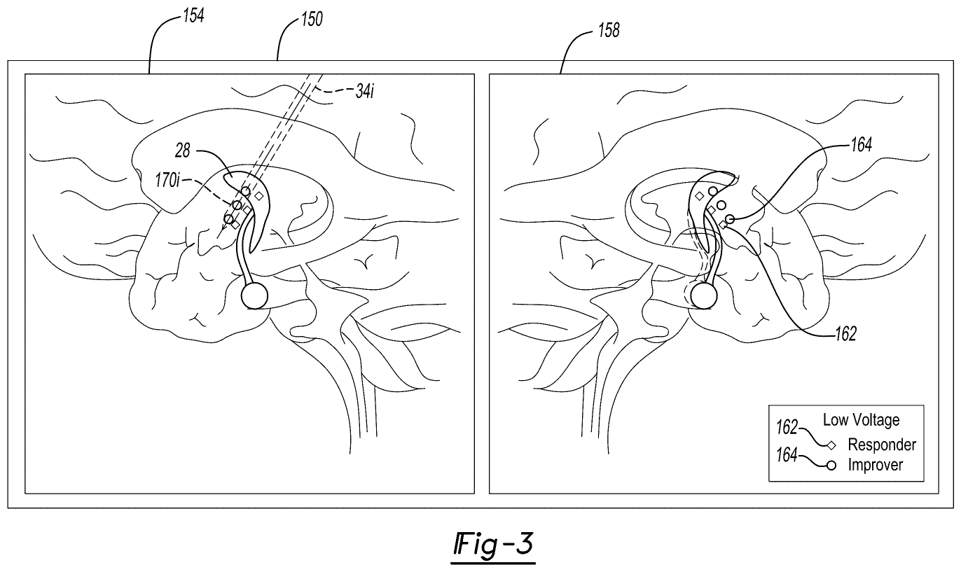

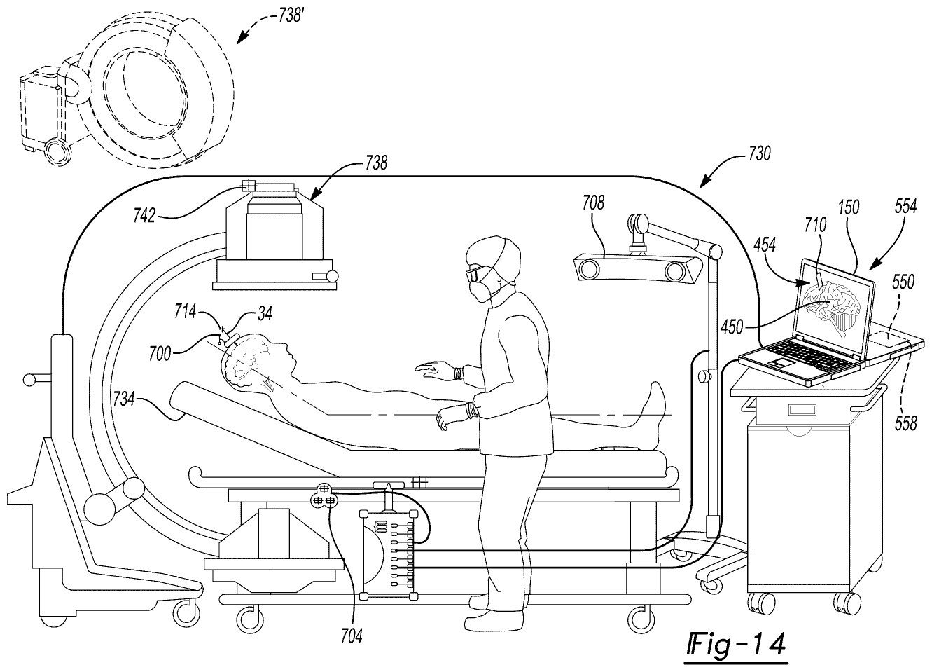

With continuing reference to FIGS. 1A and 1B, and turning reference to FIG. 2, a process 50 for identifying and selecting a placement and/or therapy to be provided to a subject 20 is illustrated. The process 50 may be carried out as instructions being executed with a selected processor module that accesses selected memory. The processor module may be a general purpose processor and/or an application specific processor module. With brief reference to FIG. 14, an exemplary processor system 554 may include a processor module 550 and a memory module 558. An output may also be made and may include a display device 150.

The process 50 can include various sub-processes and portions, including those that are optional. Thus, various portions of the process 50 disclosed herein as a part of the procedure may be understood to be a portion of the process 50 for identifying a selected position and/or therapy parameter (e.g., stimulation amplitude, pulse width, modulation, etc.). In various embodiments, the process 50 is operable to identify a selected sweet spot, which may also be referred to as a sweet spot target, sweet spot target position, and/or sweet spot target portion for placing a lead for stimulating the subject 20. The sweet spot may be understood to be a structure, portion, or position that may be a two-dimensional point and/or three-dimensional (3D) brain that may be identified within the brain of a subject, including in the image data thereof. The sweet spot target position may be based on various selections and procedures, as disclosed herein, including weighting of various features. This may, therefore, allow or include a feature data weight and/or a structure data weight. The sweet spot target position may be understood by one skilled in the art to be a position that is determined, such as based on the process 50, for positioning an implant, including an electrode thereof, to provide a therapy to the subject 20 to achieve a selected result. Thus, the sweet spot target position may not be determined separate from the process 50, but as a result of the process 50. The selected result may be an optimal result for the subject 20 that is determined by the process 50. Nevertheless, it is understood that various portions of the procedure 50 may not be required and are optional as discussed herein.

The process 50 may include, optionally, a predetermination of an optimality space (POS), also referred to as optimality data, in block 54. The optimality data (e.g., one or more) may be or include a predetermination of an optimality space and may include various processes and actions, such as identifying selected structures (also referred to as a structure space also referred to as a structure data (e.g., one or more)) in sub-block 58 and/or identifying selected features (also referred to as a feature or dimension space also referred to as a feature data (e.g., one or more)) in sub-block 62. The POS 54, including the sub-portions as discussed herein, may be used to identify or select a target position that may also be referred to as a sweet spot target position for an implant or probe with the process 50.

The selected structures identified in sub-block 58 may include identifying maps of portions of a subject, including a group of subjects that are in addition to or extra to a current subject. As discussed herein, the structures may be included in one or more structure data and may have a one or more structure data weights selected therefore. Thus, current subject may be a new or current subject for which the process 50 is being executed for outputting a sweet spot and the current subject is not a subject included in the group of subjects used to identify structures. Extra or additional subjects may be those from which the POS 54 is defined. For example, a group of subjects may be averaged to identify a map of various portions of an average subject, such as anatomical structures in a brain. In various embodiments, such maps may be referred to as an atlas and may be identified for use relative to a specific patient or subject, such as through registration or warping to the current patient or subject, such as discussed further herein. The atlases, therefore, may be generalized or formed from a general population. The maps may also be features that are identified in the feature space that are weighted in determining the target, also referred to the sweet spot. The weighted feature or feature space may be referred to as a feature space or data weight or weighting.

Further selected structures in block 58 may include identified fiber tracks or tracts within the atlas. The structures may include identified networks, which may include one or more tracts. The tracts may be identified in any appropriate manner. For example, various fiber tracks may be identified within the atlas using selected techniques such as diffusion data and selected analysis thereof to identify a fiber track. In various embodiments, post-mortem studies may also be performed to assist in identifying selected fiber tracks within a population.

The selected structures in sub-block 58 may also include fingerprints that are used to identify portions within a subject. Generally, the fingerprint is defined as a set (sphere, of other geometric volume) containing various brain structures (e.g., areas or fibers). The exact composition of this set is a specific fingerprint. These fingerprints may be used to identify selected portions or sub-portions of an anatomy, such as a selected portion of the brain including the thalamus 28. For example, a determination of an anterior portion of a thalamus, such as the ANT 30, may be a fingerprint that may be predetermined and saved as a selected structure. The fingerprint includes information and data that relates to identifying the specification position.

Selected features 62 may include attributes of a therapy and may be selected upon positioning of a probe or implant, parameters of operation of a probe or implant, or other selected portions. The features are in addition to the structures that may be identified or selected in the POS 54. The selected features may relate to outcomes of a therapy in a subject or dynamics of the therapy or the outcome in the subject.

Selected features may include selected outcomes such as determined best outcomes, optimal outcomes or the like. Selected outcomes may also be identified or determined along a spectrum or finer than simply best or optimal, such as in any appropriate range between undesired and optimal. Selected outcomes may also include a presence or lack of a side effect, such as identified as an un-selected outcome. A selected feature in sub-block 62 may also be a stability of outcomes. A stability of outcomes may include an outcome that is achieved or maintained over a selected period of time, such as over more than one year, more than three years, more than five years, or the like. A stability of an outcome may, therefore, be used to identify an outcome that may be achieved and maintained over a selected period of time (e.g., life time of stimulation the subject or patient). Dynamics of outcomes may also be a selected feature. Dynamics of an outcome may be the rate (i.e., change over time) at which an outcome is achieved from an initial point to the selected outcome. Dynamics may also include the rate of change, thus dynamics may include a high or fast dynamic change and a low or slow dynamic change in the outcome. Dynamics of outcomes may include an outcome that is achieved for a certain period of time and then a change to the outcome. Accordingly, a stability of outcome may be the ability to achieve a selected outcome while a dynamic of an outcome may be the speed or change over time to the stable outcome. Selected features may also include stimulation amplitude dependency, where a selected outcome is based upon a required input. Thus, the feature space or data may also relate to or include feature space outcomes or identified outcomes related thereto.

As discussed above, a therapy provided to the brain may be a neuromodulation. The neuromodulation may include a stimulation to the brain, as discussed herein, may be a stimulation including an electrical stimulation, such as an application of a voltage, amperage, etc. As used herein, stimulation therefore, including electrical stimulation, may refer to neuromodulation. Thus, neuromodulation which may be application of an electrical feature (e.g., amperage, voltage, etc.) may be referred to as a stimulation and may result in either or both of a excitatory or inhibitory response in a brain network. The neuromodulation, such as the electrical stimulation, may include a selected voltage and/or stimulation current applied with an electrode contact located on a DBS lead, amplitude such as in amperage (e.g., milliamps (mA)), duty cycle, varying voltage and/or stimulation current over time, or the like. Accordingly, the amplitude may be selected to be a selected amount, such as less than 3 volts, greater than 3 volts, or a selected duty cycle. Alternatively, and or in addition, a current may be selected and/or varied such as greater than or less than 1 mA. Therefore, a feature may be based upon an amplitude required to achieve the selected feature, such as a selected outcome or a stability of outcome. Further, a pathway activation may be a selected feature including the strength of the activation based upon a placement of a lead, an activation based upon an amplitude, or the like. As discussed herein, a stimulation may be determined with a voltage and/or a current, thus discussion herein to voltage will be understood to refer to a current that is selected for therapy as well and/or alternatively.

Various predetermined features will be discussed further herein that may be determined or predetermined in block 54. These predetermined optimality space (POS) features may then be saved in block 66. Saving the POS in block 66 is also optional and may be selected based upon various features or procedures. Nevertheless, by saving the POS in block 66 the POS may be accessed and recalled for further selected procedures, such as a procedure on a current subject or a subject from which data is acquired as separate from the data used to determine the POS on block 54. It is understood, however, that patient data may be used to assist in determining the POS in block 54 but still may be saved in block 66 and recalled for a selected procedure.

Accordingly, the POS may be recalled in block 70. Recalling the POS in block 70 allows the predetermined optimality space to be evaluated and/or applied to a current subject for a selected procedure. The subject, therefore, may be selected after the determination of the predetermined optimality space, but may also include any subject to which the predetermined optimality space is applied for a selected procedure. The selected procedure may include identifying a position for placing a DBS lead, a parameter for operating the lead, or other appropriate portions of a procedure.

The recalled POS in block 70 may be used to identify various features and plan a procedure relative to the subject 20. The subject 20, therefore, can also be imaged to assist in the registration and planning of a procedure. The subject may be imaged in any appropriate manner such as with MRI, computed tomography (CT), ultrasound, or other appropriate imaging techniques. The image data of the subject may be acquired and/or accessed (e.g., recalled with a processor module from a memory system) in block 80. It is understood that the image data may be acquired of the subject at any appropriate time, such as prior to a procedure, immediately before performing a procedure, or at any appropriate time.

Further, the acquired subject data in block 80 may also and/or alternatively include prior procedures, identification of various features in the image data by a surgeon or other appropriate individual, and the like. For example, non-image data may include a specific diagnosis or prior procedures. Subject data may further include specific biographic or demographic data, such as age, genetic markers, etc. Other data regarding the subject may include: onset zone of epilepsy in the brain of a specific subject, type and severity of the epilepsy experienced, medication used, duration of the disease. All or any number of the data may be used. Subject data may also include other types of data, such as electrograms. This subject data from block 80 may, optionally, be integrated into eh POS 54 via path 83. Thus, a selection may be made to include the subject data in the POS 54 for further analysis.

Further, a plurality of image data sets may be acquired. If more than one image data set is acquired, a co-registration of the acquired image data sets may occur in block 84. It is understood that a co-registration is not required, but may be selected if multiple image data sets, potentially providing complementary information, are acquired of the subject. For example, an MRI and a CT data set may be acquired of the subject. The MRI and the CT may be co-registered to one another prior to further analysis. Such co-registration may allow for ensuring planning is performed relative to identical locations or portions within the image data of the subject. Co-registration may also be useful or essential if images are acquired at different periods in time (e.g., Pre-Operative and Post-Operative).

Image data of the subject acquired in block 80 may be registered or warped to the POS in block 88. The warping of the subject data to the recalled POS may be performed at any appropriate manner, such as those discussed further herein. The warping allows various structures in the subject data to be registered or co-located with structures in the POS. For example, the thalamus may be identified and co-located or registered between the POS data from block 54 and the acquired subject data in block 80 due to the warping in block 88. Thus, the information regarding the predetermined optimality space may be registered and used to identify spaces or positions within the subject data due to the transformation in block 88. It is also understood that the Optimality space may be warped or transformed to the subject data.

With the subject data warped to the predetermined optimality space in block 88, a user may, optionally, select a lead type in block 92. The lead type selected in block 92 may be any appropriate lead type. Lead types may include leads with selected types of electrodes or electrode contacts, such as a single electrode, multiple electrodes, unifocal electrode, or multifocal electrodes. In various embodiments, single or multiple electrodes may include an implant that includes a single electrode positioned on the implant and a multiple electrode implant may be implanted that includes multiple electrodes. The electrodes may be positioned on the implant in an appropriate manner, such as at a tip, at a tip and a position proximal to the tip, or at a tip and multiple positions proximal to a tip, or at multiple positions proximal to a tip of the implant. It is understood by one skilled int the art, however, that the electrodes may be positioned at any appropriate location along the lead such as the lead noted above.

Further, the electrodes may be unifocal or multifocal. That is, each of the electrodes may provide or direct a stimulating in a single direction or in multiple directions. In a multiple-direction electrode, the multiple or one or more multiple directions may be selected, based upon the type of electrode.

Nevertheless, the selection of the implant type in block 92 may allow for a selection of implants, such as a lead, that includes one or more electrodes or electrode contacts and/or one or more foci of the electrodes or electrode contacts for providing stimulation to the subject. The subject, therefore, may be stimulated with the selected implant once the implant is positioned within the subject. Thus, the implant may be positioned in the subject and during programming, the one or more electrodes may be selected and the one or more foci may be selected for providing stimulation to the subject. Thus, the process method 50 may be used to identify an appropriate electrode placement of a lead implant with one or more electrodes and/or orientation based upon a plurality of foci or a single focal for the electrodes on the implant.

After selecting an implant in block 92 and/or for example, concurrently therewith, a target may optionally be selected in the subject data in block 96. Selecting a target may include selecting a position within the subject data for positioning at least one of the electrodes of the implant. For example, as discussed above, the target may include an anterior nucleus of the thalamus, also referred to as the ANT. Other appropriate positions may be selected for a target within the subject. Nevertheless, for example, the ANT may be selected for further analysis relative thereto. It is understood, however, that the target selection may also be a first or initial selection in an iterative process and/or optional as noted above.

Initial weights may be selected for the various features, such as including structures in the structure data, in the of the POS in block 98. The feature or structure data weight(s) may include a selected relative weight or importance of each feature include in the POS. The weight may be selected to be zero, having no weight, and a selected value greater than zero. Each of the weights may be considered in the process 50 for evaluating and determining a sweet spot target position. The weights assign a relative importance to each of the structure and feature spaces for determining the sweet spot target position. The sweet spot target position, therefore, is the position where the implant 34 (including the electrode 170) is selected to be positioned based on the weighted features of the POS 54. Thus, the sweet spot target position may vary for different patients and/or based on selected of weights. It is understood, however, that the sweet spot may also refer to or include a geometric structure or volume that is to be stimulated. The weights selected in block 98 may be initial weights and/or may be varied as discussed herein. The weights may also be manually and/or automatically selected.

An evaluation of the recalled POS and the acquired subject data and/or the selected implant is made in block 100 based on the weights selected in block 98. The evaluation may also include the target if selected in block 96. The evaluation of the selected target with the recalled POS and selected implant allows for the process 50 to provide an evaluation of the implant at the selected target. As noted above, the POS includes various selected structures and selected features that may be analyzed relative to the selected target or target position within the subject for providing a therapy to the subject. The evaluation of the selected target with the recalled POS and selected implant may include a combination or combining the features and the structures of the POS by weighting them at the selected target and/or based upon the selected target.

As noted above, again, the selected structures and selected features may be based upon a selected result that may be predetermined as an optimal therapy on a subject or on a plurality of subjects. Accordingly, an evaluation of the selected target with recalled POS based upon selected weights thereof is made in block 100. The evaluation may allow for a determination of a target and/or an evaluation of a therapy provided at the selected target. As discussed herein, the weights of the POS may be selected to determine a sweet spot target position and/or determine a therapy at a selected target.

The evaluated recalled POS with the acquired subject data may allow for a rendering of the selected target for the selected weighting of the POS in block 104. The rendering may include various visualizations that may be used by a user or the process 50 to determine the sweet spot target position. The rendering, for example, may include a graphical representation of target location in the subject image data.

The rendered evaluation may be displayed in block 108. The displayed rendering may include various renderings, such as a graph of dynamic response, a display of an activated region or portion of the subject, a display of an activated brain network tract of the subject, a display of activated portions or regions relative to the subject, a time varying aspect of the results of the therapy, or the like. Various visualizations will be discussed further herein and may be displayed for evaluation or viewing by user. The displayed visualization may assist the user in identifying an appropriateness of the selected therapy for the subject. In various embodiments, the display may assist the user in determining an optimality of the sweet spot target position and/or associated therapy. Thus, the displayed visualizations may be displayed and/or altered by the user for a determination.

The displayed visualizations may also allow the user to determine whether an adjustment of various aspects, such as weights of the recalled POS should be made in block 112, which may include brain network interactions. An adjustment of the weights, also discussed further herein, may allow for a user to determine or alter a type of therapy to the subject to assist or determine a selected outcome based upon the therapy. A determination that an adjustment is to be made allows the process 50 to follow a YES path 116 to optionally select a target in block 96 or weights in block 98 to evaluate the selected target with weights of the recalled POS in block 100. It is understood that the YES loop 116 allows for an adjustment of the weights and therefore the evaluation in block 100 is based upon the last entered weights either initially and/or after the adjustment in block 112. The evaluation may then allow for a rendering and a display in block 104 and 108, respectively.

If no adjustment is selected, a NO path 122 may be followed to an output in block 126. The output may include any appropriate output. For example, the output may be one or more visualizations, as discussed herein. In various embodiments, the output in block 126 includes the position of the sweet spot target. The output may include a graphical representation of the position of the sweet spot target position superimposed on an image of the current subject with a display device. The output as the position of the sweet spot target position, therefore, relates to a position within the subject 20 for placement of the implant 34. The output, according to various embodiments, is discussed further herein. As noted above the sweet spot may also refer to or identify a region to be stimulated during therapy.

The determined sweet spot target position is the position of an implant and may include one or more electrodes and may be output by the system for guiding a procedure, as discussed herein. The sweet spot target position is the position that is determined to achieve the result of the weighted features from the POS 54 based on the process 50. In other words, the sweet spot target position is the position that is determined by weighting the features from the POS 54 based on the process 50 to achieve selected outcomes. Thus, the sweet spot target position output in block 126 may be different for each subject and may vary based upon the weights assigned to the POS 54, as discussed herein.

The process 50 may then end in END block 130. In ending the procedure in block 130, the user may select a final position for the implant, and various programming for the device, such as with the stimulator 36. Therefore, the ending of the process in block 130 may not end a procedure on the subject, but may end at the process 50 for planning and selecting an implant and position and selected programming for the implant within the subject.

The process 50 may include various inputs that may be identified and determined by various users over time and be extra to a current subject. For example, the POS may be based upon a plurality of procedures prior to a current procedure to allow for identifying and evaluating various structures and features and subjects. A study of the various prior subjects may assist in determining an outcome based upon a placement of a lead and/or therapy applied with the lead. The prior subject data, which may also be referred to as extra subject data, may allow for the determination of the POS 54. Therefore, the POS 54 may be used in the process 50 to evaluate an implant procedure on a current subject.

The various structures and features may be weighted individually according to a selected process to achieve a selected outcome. As discussed further herein, for example, a placement of a lead may achieve a selected outcome but not a selected stability of outcome and/or a selected dynamic of outcome. Accordingly, the placement of the device may be varied or moved to determine a different dynamic of an outcome. Similarly, with a lead with more than one electrode or electrode contact providing therapy with different ones of the electrodes effectively “moves” the lead. A user may adjust a weight regarding a dynamic of an outcome, a weight regarding a type of outcome, a placement, or the like to achieve a selected therapy outcome with placement of the lead. Based on the process 50, a user may determine an optimal placement based upon a selected outcome that may be specific relative to a specific subject, such as a current or specific patient. Altering weights may also affect strength of one or more brain network connections and can be considered as the resulting interaction strengths (balance) between brain networks can be evaluated by e.g.: the type and intensity of side effect of stimulation not only evoked in the ANT area, but also over long distances in the brain through brain networks, the strength of the brain network interactions can change over time (Brain plasticity), and/or the dynamics of therapy changes (fast: over hours or days) or e.g. over months (1-3 months). Not to be bound by the theory, but regarding a time period of months a change in electrode property may occur as connective tissue forms around components of a lead.

The POS including the selected structure 58 may include structures that are selected or identified in a plurality of subjects and may be used to generate an atlas or a map. The atlas or map may be based upon identifying or studying a plurality of subjects having implants positioned therein for therapy. These structures may include the thalamus, or other appropriate structures or identifiable structures in the anatomy. The structures may be identified based upon study or evaluation of selected image data, such as MRI image data, diffusion data, or other appropriate data. The maps may include identification of structures in a plurality of individuals that are combined together into a map or atlas.

The atlas may include any selected atlas such as the “Atlas of the human brain” atlas as disclosed in Juergen K. Mai, Milan Majtanik, and George Paxinos, editors. Atlas of the human brain. Elsevier AP, Amsterdam, 4. ed. edition, 2016. The atlas may be of and/or include subcortical space which may be warped or registered to a MNI space. All used images, including an atlas may be warped to a particular MNI space, allowing multiple images, also from multiple patients to be compared. MNI space is a kind of averaged image and is a stochastic space showing the statistical likelihood where various features (e.g., brain structures or e.g., a lead with multiple electrode contacts) are located with respect to each other. The atlas may be saved and used for the process 50 and may be also updated based upon evaluation of a plurality of subjects over a period of time.

The selected structures 58 may also include fibers within an anatomy, such as fiber tracks within a brain. The fiber tracks may be identified based upon image analysis, such as diffusion analysis. It is understood that any appropriate type of image analysis may also be used to identify fibers. Further, in various embodiments, the fibers may be identified based upon a dissection of an anatomy.

The fibers may be used to identify various structures in the anatomy that are connected together (Human Connectome) or may be connected together. In various embodiments, a stimulation of a fiber may stimulate both a selected structure and a second or connected structure. Such a known connection allows simulation at or near a fiber that may stimulate an area larger or structures in addition to those that are immediately adjacent to an electrode. The fibers and areas of stimulation due to stimulating at or near a fiber may be included in the selected structures.

Similarly, fingerprints may be identified that cause stimulation of a plurality of positions or structures within the anatomy. The fingerprints may identify one or more fibers and one or more structures that may be stimulated based upon a position and a focus or foci of the electrode. The fingerprints may also be used to identify areas that may stimulate a plurality of portions of a subject without requiring placement of a plurality of leads. Further, the fingerprints may identify areas or positions within a subject that are likely (e.g., greater than 50% probability) stimulated which may not be selected for various procedures or diagnoses.

Thus, the POS may include an identification of a plurality of structures, as illustrated in FIGS. 1A and 1B. The structures may be used to identify possible targets for positioning the implant 34 and/or one of more electrodes 170 thereof. The target structure or portion of a structure, such as the ANT 30, may be used to provide a position for the implant and the application of a therapy, as discussed further herein. A location of the implant 34 may be weighted, such as selecting a placement near a selected structure and/or near a selected fiber to achieve a selected result. Nevertheless, as noted above, a selected placement may stimulate and/or neuromodulate (i.e., affect) a non-adjacent area in the anatomy, such as an area away from the selected target, and may lead to an outcome or possible outcome that is not selected for a particular subject. Therefore, the use of the structures may also assist in identifying a target and may be used to weight the target in addition to the features, as discussed further herein.

Briefly, a target 140 may be illustrated superimposed on image data, such as the images illustrated in FIG. 1A and 1B, to assist the user in defining a position for placing the implant 34. During the weighting and adjustment sub-process 127, after an initial weight is evaluated, the target may be moved after adjustment in block 112 following the YES path 116. Therefore, one skilled in the art will understand that the target 140 may be updated over time, including repositioned or moved relative to an anatomical structure. Further it is understood that the target 140 may be placed relative to various different anatomical structures, such as the thalamus 28 or other structures, including a caudate nucleus 142 and/or a subthalamic nucleus 144.

As noted above, the POS 54 may also include selected features in block 62. These selected features may also be referred to as dimensions that may be analyzed in the optimality space. Again, the selected structures and selected features may be portions that are analyzed or studied in a population of patients, also referred to as extra subject data, to be used to assist in planning a procedure for a selected current patient. Thus, the various outputs based upon the selected structures and features may be analyzed and/or altered to achieve a selected result that may also be referred to as an optimal result for a selected subject.

As discussed further herein, the selected structures in block 58 and the selected features in block 62 may be combined for determining a selected plan or output for a subject. The selected structures and selected features may be evaluated individually and/or together when determining a selected output or therapy for a subject. Accordingly, while the structures and features may be discussed separately herein, it is understood that they may be evaluated in combination, such as weighting each in an additive or multiplicative manner, when determining a therapy for a subject. The therapy may include determining a sweet spot target position for an implant.

The process 50 for identifying and selecting a placement and/or therapy to be provided to a subject 20, including the output sweet sport target position in block 126 may be carried out as instructions being executed with a selected processor module that accesses selected memory. The processor module may be a general purpose processor and/or an application specific processor module. With brief reference to FIG. 14, an exemplary processor system 554 may include a processor module 550 and a memory module 558. An output may also be made and may include a display device 150. The adjustment of the weights in block 112 may also be automatic and/or manual. For example, the adjustment may require input from a user. The adjustment in block 112, however, may also be performed based on a machine learning system that is trained with selected data to adjust the weights to achieve the selected outcomes. Selected outcomes may include a subject that responds to a therapy, an outcome that is stable, and other features, as discussed herein. The optimization of the weights with machine learning can use standard techniques as for example supervised or/and reinforcement learning,

Accordingly, various selected features or dimensions may include those noted above and/or those discussed herein. For example, a selected feature may include a selected stimulation amplitude of therapy and/or other appropriate therapy. The implant may include a therapy device, such as a stimulating device which may include a deep brain stimulation (DBS) implant. These may include single or multiple electrodes as discussed above. In various embodiments, a selected stimulation amplitude of therapy may be a stimulation identified in volts and/or amperage. For example, a therapy that is three volts or less. It is understood, however, that therapy may be provided at greater than three volts. Amplitude of 3 volts or less may include about 0.1 to about 1 volts, about 0.5 to about 2 volts, about 1.5 to about 2.5 volts, or other selected voltage ranges. Nevertheless, a user may select to have a desired outcome or therapy to have a voltage of three volts (3V) or less. Stimulation may also be referred to as a current, including such as measured in milliamperes. Thus, neuromodulation may include a stimulation that may be provided or understood as a current to the subject.

Initially, a patient or subject may respond to a DBS in one or more of various manners. For illustrative purposes, in the subject of disclosure, a responsible subject may be at least one of and/or a blend of a responder, an improver, or a no benefit. A no benefit generally relates to a lack of selected change in a diagnosis or prognosis of a subject after therapy or following therapy. This may also include a worsening of an outcome of the subject 20. All three conditions may be a balance between desired improvements (e.g. in epilepsy: seizure frequency or severity reduction) combined with acceptable or non-acceptable side effects of neuromodulation stimulation.

For example, in epilepsy a no benefit may include a no change in a duration or frequency of seizures and/or an increase in in rate or frequency of seizures. Other responses may include an improver that improves a prognosis after initiation of therapy. An improver may see a decrease in a frequency or duration of seizures a selected amount, such a reduction of up to about 50 percent. For example, in a diagnosis of epilepsy, a change in seizure rate from 2 over 30 days to 1 over 30 days may be identified as an improver. A responder may be a subject or patient that has a greater than 50 percent change in prognosis after therapy has begun. For example, a patient that has a change in number of seizures from 2 every 30 days to 1 every 90 days or none during a follow-up period may be identified as a responder. Accordingly, patients or subjects may be identified as being in one of three categories including a subject that sees no benefit, a subject that improves or is an improver, or a subject that responds or is a responder. It is understood, however, that other types of outcomes may be identified or less than three.

Turning reference to FIG. 3, the selected structure may include structures such as the ANT 30. The POS may further include other structures such as the mammillary body (MB), or other appropriate structures such as ANT surrounding brain structures, including the Mammilo thalamic track. As illustrated in FIG. 3, for example, an image may be displayed on a display device 150. The image may include an image of a left portion of the brain 154 and an image of a right portion of the brain 158. The two images may be images of the same atlas based on a plurality of subjects, as discussed above. Other structures may also be identified in the images, such as tracks or fingerprints. It is further understood that other appropriate structures may be illustrated, as selected by the user.

Illustrated on the images 154, 158, or only a single one of the images, may be one or more target positions that can be identified associated to various prognoses or outcomes based on target position. The prognosis or outcome may include an improver region, which may include a location that is signified by a circle or a responder region which may include a location that is illustrated by a diamond. The various positions for the responder 162 or the improver 164 may be illustrated on a display 150. The positions may be related to a specific amplitude, such as less than 3 volt amplitude. Therefore, the user may view various positions and identify or determine outcomes related to the positions, such as responder 162 or improver 164. The selected position based upon the low voltage and the response thereto may be selected or weighted by the user or a selected system, such as a machine learning system as discussed further herein, for determining a position and programming of a therapy to the subject. Again, volts or voltage may also refer and/or relate to current of electricity within the subject from the device for therapy.

The various positions for the responses, such as the responder 162 or the improver 164 may be based upon various input, such as the predetermined optimality space is based upon one or more prior performed therapies and outcome responses thereto. Therefore, the display 150 may display one or more positions for an implant. The position for the implant may include a position of an electrode on the implant 34 and may include a plurality of electrodes along the implant. Accordingly, one implant may cover more than one of the positions, for example, as illustrated in the image 154.

A graphical representation 34i of the implant 34 may be on the display 150. The graphical representation 34i may include various additional graphical representations, such as graphical representations of the electrodes 170i. The representation of the electrode 170i may relate to one or more electrodes 170 that are formed or placed on the implant 34. For example, as illustrated in FIG. 1A, the implant 34 may include a tip electrode 170′ and three ring electrodes 170″, 170″′, and 170″″. Each of the electrodes may also be segmented to allow for a plurality of foci each of the electrodes 170. Thus, the display 150 may illustrate the electrodes 170i and the implant 34i. A user or the system 50 may identify a position of one or more electrodes that may be placed at one or more of the predetermined positions for responder or improver 162, 164.

With continuing reference to FIG. 3, various positions, such as the responder 162 and the improver 164 positions, may vary over time. It is understood by one skilled in the art, a therapy may be provided to a subject and the therapy may cause changes to the anatomy of the subject. For example, in the brain, various pathways may become altered and/or normalized to a selected input. In other words, the therapy may cause balances between brain networks. The brain physical feature anatomy may stay the same, but adaptation in strengths of brain networks may change with therapy. Therefore, over time the improver and responder positions may alter and/or alter their outcome. For example, a responder position may become an improver position over a selected period of time. Thus, the positions of responder and improver may also include information regarding time-varying aspects thereof. The time-varying aspects may also be determined based upon a predetermined evaluation of selected data.

The time-varying aspects of the positions may be included as a portion of the amplitude feature. Further, the voltage applied may also vary over time to achieve the responder or improver aspect of the position. For example, a change from a high to a low voltage or vice versa. A change from a low to a high voltage may change the outcome from an improver to a responder and/or vice versa. The amplitude dependent feature, therefore, may include these aspects in addition to a static position and may be included in the POS 54. It is understood, however, that other aspects may cause time varying aspect to the outcome. Again, volts or voltage may also refer and/or relate to current of electricity within the subject from the device for therapy.

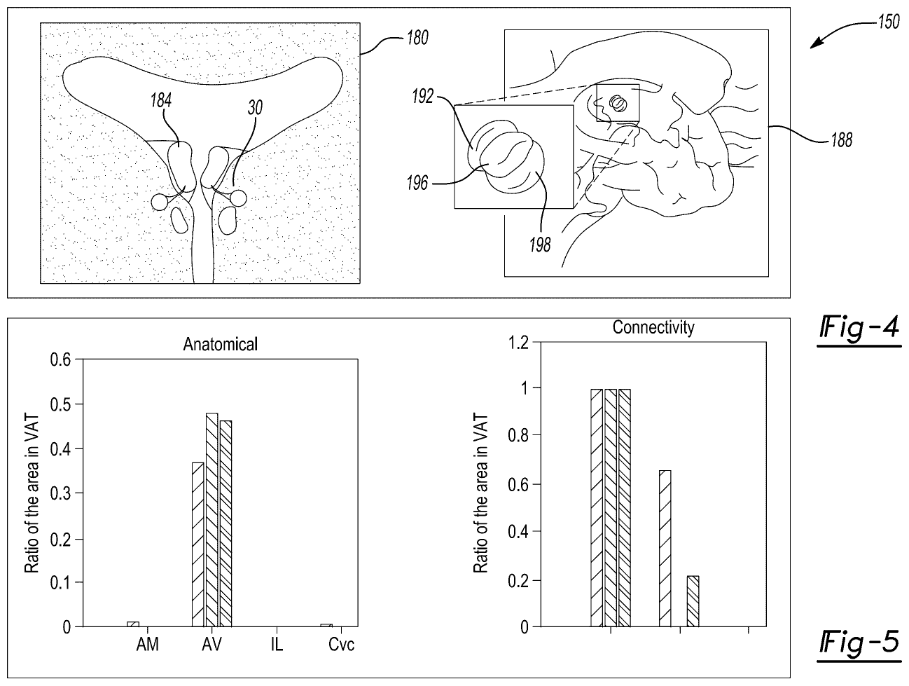

Turning reference to FIG. 4 and FIG. 5, an additional feature that may be analyzed or included with the POS 54 is a stimulation outcome score. The stimulation outcome may include a determination or evaluation of an outcome based upon a volume of activation which may include a volume of activated tissue (VAT). A determination of a VAT and a related response of a subject may be determined based upon the position and selected therapy, such as amplitude within a plurality of subjects when determining the POS. As discussed above, the optimality feature of the VAT may then be applied to a current subject to assist in determining and planning a procedure. The VAT is generally a volume of tissue that is stimulated or activated in the subject 20 with a selected stimulation. The volume may be represented on a display with a graphical representation superimposed on an image of the subject 20 and identify one or more voxels of portions of voxels in the image.

With initial reference to FIG. 4, for example, an image 180 may be displayed. The image 180 may be based upon an atlas or predetermined plurality of subjects. The atlas image 180 may include various portions that are identified within a subject or plurality of subjects, such as the ANT 30. The ANT 30 may be illustrated relative to the image 180 based upon a known or mapped location thereof. A volume of activation 184 may then be illustrated relative to the ANT 30. The volume of activation may vary depending upon a position of an implant, such as the implant 34 discussed above and a selected therapy or programming with the implant. For example, a selected higher amplitude may increase a VAT while a lower amplitude may decrease the VAT. The displayed VAT may illustrate the VAT relative to various known or defined anatomical structures, such as the ANT 30.

One skilled in the art will understand that one or more VATs may vary based upon placement and/or amplitude of a therapy device. Thus, one or more VATs may be illustrated in the image 180 and/or alternative image 188. The alternative or aggregation image 188 may include an illustration of a plurality of VATs and associated outcomes. For example, a first volume 192 may be related to non-responders or no benefit. A second VAT 196 may relate to improvers. A third VAT 198 may relate to responders. Accordingly, the various VATs may be used to identify or relate to possible outcomes based upon VATs within the subject. As discussed above, the position of the implant 34 and a selected therapy amplitude, such as a voltage, may achieve selected responses. A VAT may be based upon a known anatomy, such as fiber, or the like in the subject, and a related outcome may also be known or included in the POS 54. Therefore, the VAT feature may additionally apply to determining a therapy and/or position of the implant 34 including one or more electrodes.

Additionally, the data or information in the POS may include non-graphical information which may include selected aggregation of outcomes that may be referred to herein as a “fingerprint.” Fingerprints may include anatomical fingerprinting or connectivity fingerprinting as illustrated in FIG. 5. The graphs illustrate anatomical portions or regions and whether they are within a VAT and the outcome related thereto. In anatomical fingerprinting, outcome associated fingerprinting may be applied to various anatomical portions within the subject in the POS. This may assist in defining various positions within the atlas 180 and assist in determining positioning for the implant 34. For example, in an anatomical fingerprinting the MB may be related to substantially no outcome while positioning the VAT relative to the ANT may include identification of at least outcomes related to no benefit, improvers, and responders. It is understood, however, that any appropriate anatomical portion may be determined to relate and/or not relate to any particular outcome. In addition and/or alternatively, connectivity fingerprinting may also be identified which may be used to determine connections between or relations of various anatomical portions within the subject. Accordingly, the MB identified as connected to portions that may assist in providing a selected outcome, such as a no benefit outcome, responder outcome, or improver outcome which may be different than anatomical fingerprinting and/or different from ANT volume of activations.

The fingerprinting may be used to further assist in identifying the locations or positions within a subject for positioning the implant 34 to assist in providing a therapy to the subject.

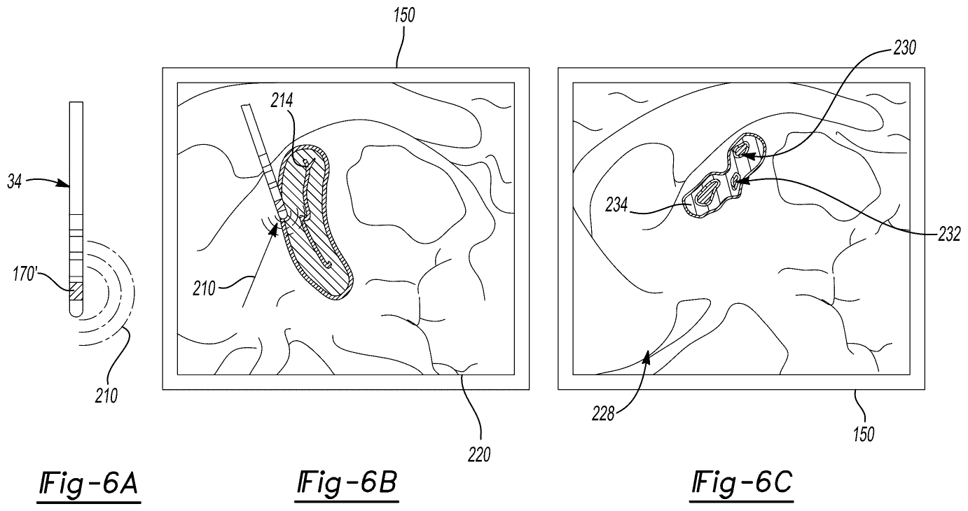

The VATs, as discussed above, are based upon an activation volume due to an activation of a selected portion of the subject or studied subjects based upon the implant 34. As exemplary illustrated FIG. 6A, the implant 34 includes one or more of the electrodes and/or electrode contacts 170, such as the tip electrode 170′. The electrode may be activated and generate a substantially spherical region (e.g., first order approximation spherical region) of activation that may be referred to or be identified as a boundary of an activated region 210. The boundary of an activated region may identify those portions in the subject that are activated by the application of the therapy from the electrode. Exemplary therapies may include a stimulation at a 4-volt amplitude, a frequency of 130 Hz, and about a 90 microsecond pulse width. The volume stimulated may be defined by the boundary 210. The simulated boundary 210 may be based upon selected features adjacent to the electrode 170′, and other constraints. Nevertheless, the determined boundary of activation 210 may be identified in the image by a graphical representation that is superimposed on the image of the subject 20 which may be displayed on the display 150. Again, volts or voltage may also refer and/or relate to current of electricity within the subject from the device for therapy.

Turning reference to FIG. 6B, the volume of activation may be understood and studied to determine a pathway activation profile (PAP), based upon the selected stimulation input and placement of the implant 34, including the electrode 170′. As exemplary illustrated in FIG. 6B, the volume of activation 210 may generate or relate to a PAP 214 that may be based upon a selected anatomy of the subject, such as determined fiber tracks, adjacent anatomy, and the like. As exemplary illustrated in FIG. 6B, the PAP may also be based upon a single activation or single electrode of the implant 34. As discussed above, a plurality of electrodes may be provided on the implant 34 and therefore each electrode may include a similar or different PAP.

The PAP, however, may be understood to activate a plurality of regions within the subject, which may be illustrated in a PAP image 220, such as displayed on the display device 150. It is understood, however, that the PAP 214 need not be displayed individually, but may be included within the process 50 to assist in planning and positioning an implant in the subject 20.

As noted above, the PAP may include an activation pathway through the subject and may include one or more foci based upon the stimulation input and/or a plurality of inputs. Generally, the foci may be at or a distance from a stimulation portion of the device 34. For example, the implant may have one or more electrodes. The electrodes may provide a stimulation that causes a VAT and a PAP. Foci of activation, therefore, need not be at and may not be in contact with the electrodes.

A mean pathway of activation may be determined based upon the PAP 214 for one or more of the electrodes and may also be defined such as a mean pathway activation image or analysis 228. The mean-pathway analysis may illustrate or determine one or more foci of activation based upon the determined PAP 214. For example, based upon a selected stimulation at a stimulation position (e.g., voxel) in the ANT region may define two foci at the border of the anterior ventral nucleus 230 and the internal medullary lamina 232. A third foci 234 may be defined at a pre-reticular zone. Accordingly, at a selected amplitude the individual foci may be identified based upon a mean-pathway activation. Again, the mean-pathway activation image 228 with the identified foci may be displayed on the display device 150 for visualization by user. It is understood, however, that such a visualization separate from a final plan is not required. Nevertheless, the identified foci may be determined for assisting and planning a procedure.

Further, foci may be determined based upon a plurality of varying or different inputs and may be determined based upon the varying inputs to assist in determining a therapy for the subject. For example, different foci may occur or be identified based upon a higher or lower voltage, a shorter or longer pulse width, or the like. Thus, the three foci 230-234 are merely exemplary and may be included in the POS 54. Other foci may be determined based upon a greater amplitude, or other parameter, or additional or alternative therapies and/or locations of the implant 34.

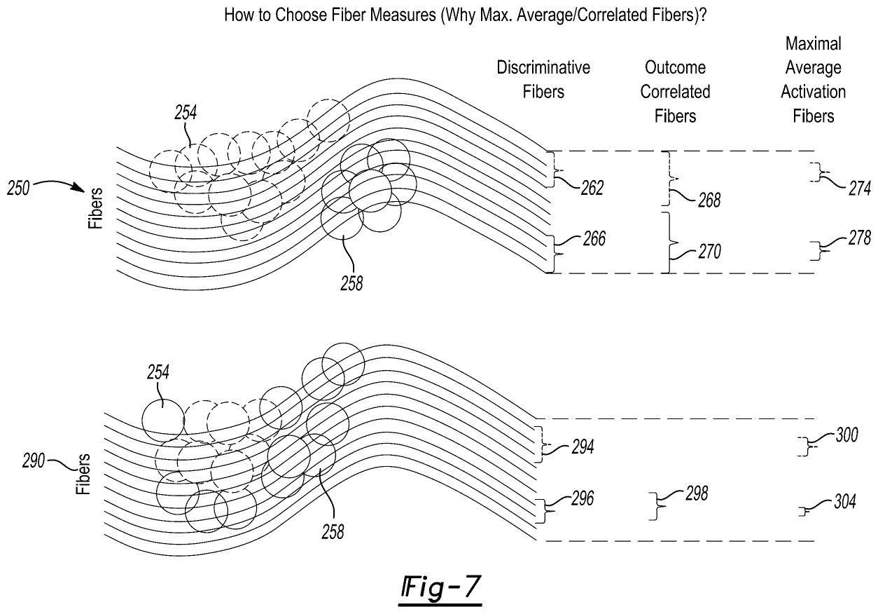

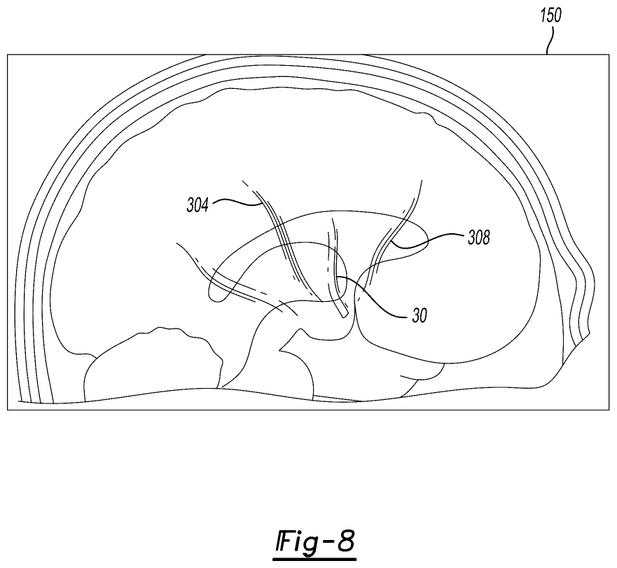

Turning reference to FIGS. 7 and 8, the outcomes may also relate to the volume of activation that activates one or more fibers, as discussed above. The fibers may be identified or determined within a subject, such as with an atlas. The fibers may identify tracks or fibers within the brain or other anatomical structure that connect more than one anatomical structure. The fibers, as is generally understood in the art, include pathways or communication paths between different areas, such as within the brain of a subject.

With reference to FIG. 7, a schematic view of various fibers is illustrated. The fibers may include a plurality of fibers illustrated as a first schematic set of fibers 250 that may include a plurality of fibers. The fibers may be activated based upon a stimulation of an area or volume, such as a VAT. The POS 54 may include information regarding which fibers are generally responding fibers when within a VAT 254. Similarly non-responsive or no-benefit responders may include fibers that are stimulated with a VAT 258. As illustrated in FIG. 7, the responder VATs 254 and the non-responder VATs 258 may overlap the same fibers at different locations. Accordingly, the different fibers may accumulate at discriminative fiber regions that include responder fibers 262 and non-responder fibers 266. This allows for a determination of outcome-correlated fibers that are responder fibers 268 and non-responder fibers 270. Finally, averaging the stimulation or results of more than one of the fibers allow for a determination of activation fibers that lead to a maximal responder 274 and a maximal non-responder 278.

As illustrated in FIG. 7, therefore, VATs can be determined and/or illustrated that are related to responders or non-responders and relate these to selected or determined fibers. This allows a determination of a number of fibers that may be determined to achieve a responder average output based upon an activation 274 as opposed to non-responders 278. In various positionings, however, the VATs of both responders and non-responders may overlap more, as illustrated in the fibers 290. Therefore, a responder VAT 254 and a non-responder VAT 258 may overlap more fibers in various positions, as illustrated in the schematic fibers 290. This leads to a determination of a fewer number of discriminative fibers. Therefore, in the second example 290, the discriminative fibers that are responders may be fewer or a smaller region 294 than non-responders 296. Further, outcome-correlated fibers may only include negative or no responder outcomes 298 and no positive responder or improvers. Finally, a maximal average fibers may be identified for those regions or fibers that are responders 300 and a different portion for non-responders 304. The first and second schematic illustrations illustrate how different VAT regions and/or fibers provide varying outcomes to subjects.

Accordingly, the schematic illustrations of FIG. 7 illustrate a plurality of fibers that may be within a VAT that is associated with either a responder VAT or a non-responder VAT at different locations. A VAT, however, may overlap fibers in certain locations that are responding fibers and non-responding fibers in other locations. Therefore, the final maximal output as illustrated schematically in FIG. 7 may assist in identifying those fibers that when activated achieve a responder or improver outcome as opposed to a not-responder outcome.

Turning reference to FIG. 8, the information may then be displayed, if selected, on the display device 150 to superimposed on a subject or atlas image. A selected number of fibers that are responder or improver fibers may be illustrated as responder fibers 304. Similarly non-responder fibers may be illustrated as 308. The fibers may be illustrated relative to selected anatomical structures, such as the ATN 30. Positions of the implant to include the respective fibers within a VAT may then be determined and/or visualized. Thus, the user may determine or evaluate the possible positioning of the implant and a VAT relative to the implant, such as based upon an amplitude of stimulation.

Again, this provides a feature or dimension that may be included in the POS to assist in determining a position for an implant and a selected therapy with the implant, such as selection of electrodes, an amplitude of stimulation, or the like. As noted above, the implant 34 may include a plurality of the electrodes 170. Therefore, achieving a VAT in one or more positions may be based upon the stimulation provided with one or more of the electrodes on even the single implant 34. Thus, the evaluation may be based upon which of the electrodes are used to provide stimulation, duration and sequencing thereof, and as similar aspects thereof.

As noted above, various features or dimensions may be used to parameterize or be identified as parameters for planning a procedure to assist in determining a position and therapy stimulation for a subject. As also discussed above, various outcomes may change or be achieved over time. Thus, the outcomes may vary over time. For example, an input stimulation may vary and benefit of a subject may vary over time. Briefly, for example, a subject may initially receive no benefit and then later become an improver or responder to a selected therapy.

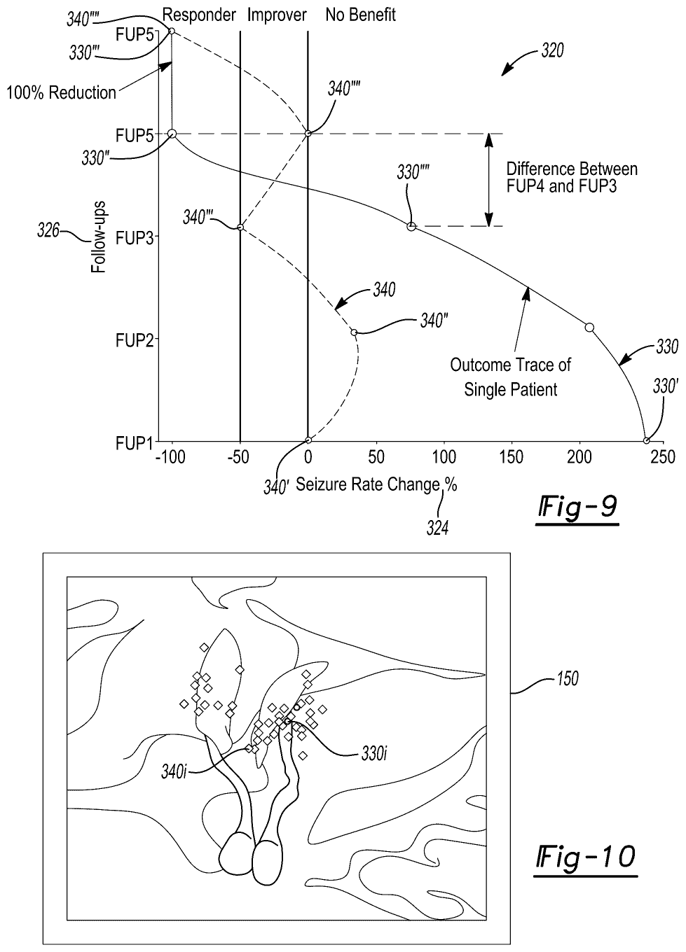

The time-varying aspect of the therapy may also be known or determined in the POS. For example, a change of an outcome to a patient may be identified over time which may be based upon a stimulation amplitude, a position of an electrode, or other features as discussed above. Turning reference to FIG. 9, the POS includes an outcome of a subject that changes over time based upon a selected number of other features, such as position and amplitude of stimulation. The outcome may be known and represented on a graph 320 as illustrated in FIG. 9. The graph may include an x-axis 324 that represents a seizure rate (e.g., number per day, number per month, number since a last clinical visit), for example for a subject 20 diagnosed with epilepsy to be treated with the therapy. A time, which may be identified as a follow up period or number, may be illustrated on a y-axis 326.

While it is understood that a plurality of subjects may be included in the POS, two exemplary subjects are illustrated in the graph 320. In a first subject a single trace 330 illustrates a time-varying outcome of the single subject. For example, in a first follow up, a seizure rate may change to be greater than 200 percent of the baseline (i.e., prior to initiation of therapy). Over time, however, as illustrated in graph 320 at various follow ups, the percent change of seizures relative to the initial state may slowly decrease to become less than or nearly 100 percent reduction at follow up for 330″ and may continue at 100 percent reduction at the next follow up 330″′. Accordingly, the various information regarding the patient 330 including the position of the lead, amplitude, activation traces, VATS, etc. may all be included in identifying or illustrating or understanding the patient trace 330.

The rate of change or dynamics of outcomes may be understood based on these feature spaces. The dynamics may be that an increase in seizures may occur, but a substantial reduction or elimination of seizures over a selected period of time may also be understood. Further, the rate of change may also be viewed in the trace 330. For example, the rate of change from the first follow up 330′ to the third follow up 330″″ may be relatively slow. A rate of change from the third follow up 330″″ to the fourth follow up 330″ may, however, be substantially increased relative to the initial rate of change. The rate of change can also be used to evaluate the effect of changes made to possible relevant parameters (POS). and can become a patient specific therapy learning curve which may, in turn, help to further improve the therapy in a specific patient. Therefore, the rate of change based upon the patient to which the trace 330 relates may also be used to understand a rate of change and when the change of outcome may occur.

A second trace 340 is also included on the graph 320. The patient trace 340 may vary over time and initially have no change at a first follow up 340′, a slight and gradual worsening to the second follow up 340″, a slight improvement to an improver status at a third follow up 340″′, followed by a worsening at a fourth follow up 340″″. Following the fourth follow up 340″″, however, the second trace 340 may reach a responder status including a substantially 100 percent reduction in seizures at a fifth follow up 340″″′. The patient for the trace 340 may also have various known or included features of structures, such as an amplitude and placement of the implant that is allowed to understand the rate of change as illustrated in graph 320.

The graph 320 may illustrate the rate of change of one or more subjects and may be understood to be used to identify a change in a subject's symptoms after an implantation. In various embodiments, the graph or data 320 may include a plurality of subjects and related dynamics of outcomes. Thus, the user may be able to identify or understand possible outcomes, change in outcomes, or rate in change in outcomes of a subject based upon a selected position of an implant and information regarding an implant therapy or placement. Thus, the rate of change or stability of change of the outcomes, as exemplary illustrated in the graph 320, may also be used to understand or identify possible placements and programming of an implant.

The graph 320 may be used to illustrate a specific subject change over time. In a similar manner, a graphical representation of implant positions may also be generated or identified, as illustrated in FIG. 10, that illustrates outcome dynamics. The display device 150 may display an image and positions of the implant 34, or particular electrodes of the implant. The placement for the subject of the trace 330 may be identified as a position representation 330i. A position placement for the implant 34 for the subject related to the trace 340 may be illustrated as the position 340i. Accordingly, a user and/or the system executing the process 50, may know the position of the implant or electrode of the implant related to the specific traces 330, 340. Thus, dynamics of outcomes such as the type of change, rate of change, and position of the implant that led to the rate and type of change may be known or for use by the system in identifying a time-changing application of therapy and outcome of the subject.

A further dimension of the POA 54 may be a dimension or feature that identifies the position in the subject that includes only stable responders. As noted above, a stable responder may be one that includes a minimal amount of change of outcome over time. While the features illustrated in the graph 320 includes an evaluation of a change over time and a rate of change, an identification of the position of the implant that achieves a stable response may be included in a further feature and may be identified as a stimulation outcome score for a stable responder. A stable responder may include a responder to a therapy whose outcome changes less than 50 percent over three consecutive follow ups after a selected period of time, such as after a first follow up period. Thus, the POS 54 may include identification of a position and other features, such as amplitude, that is understood to achieve a stable response in the subject. The stable response may be identified in the POS and may be selected or useful for identifying a position and selected therapy to achieve a stable response in a current (including a new) subject.

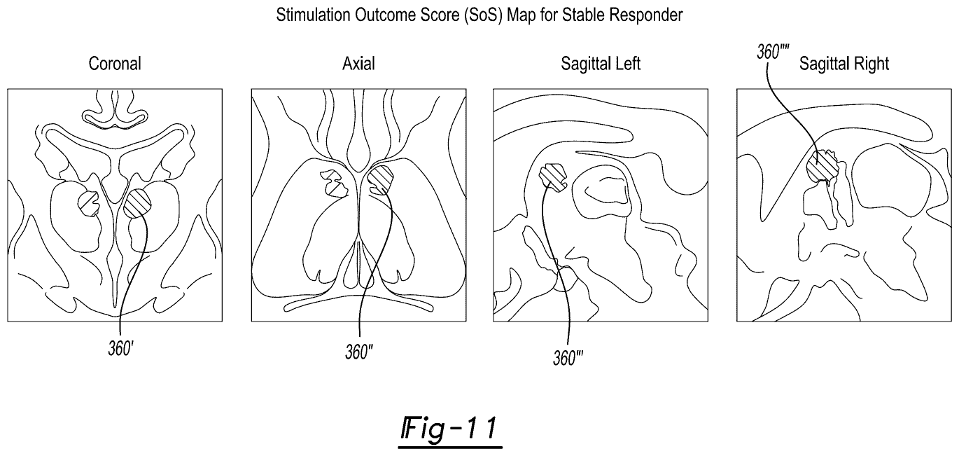

With reference to FIG. 11, a schematic illustration of a position to achieve a stable response is illustrated as 360′, 360″, 360″′, and 360″″ and may be identified in various views of the subject. The stable responder position may be identified and, in various embodiments, illustrated for the user. It is understood, however, that the stable responder position 360 may be identified in the images, such as an atlas in the POS 54 to be used for assisting and determining a position of the implant to achieve a stable response in a current subject. The stable responder position, therefore, may be used to assist in evaluating a possible position of the implant for a stable response in a current subject and may be used or weighted to achieve a selected stable response and/or an amount of change, such as a responder or an improver to a therapy.

The stable responder position 360 may be identified not only for a position, but also based upon a selected therapy, such as a stimulation. Thus, the feature may be included in the selected features 54 in the POS for use in the process 50, as illustrated in FIG. 2.

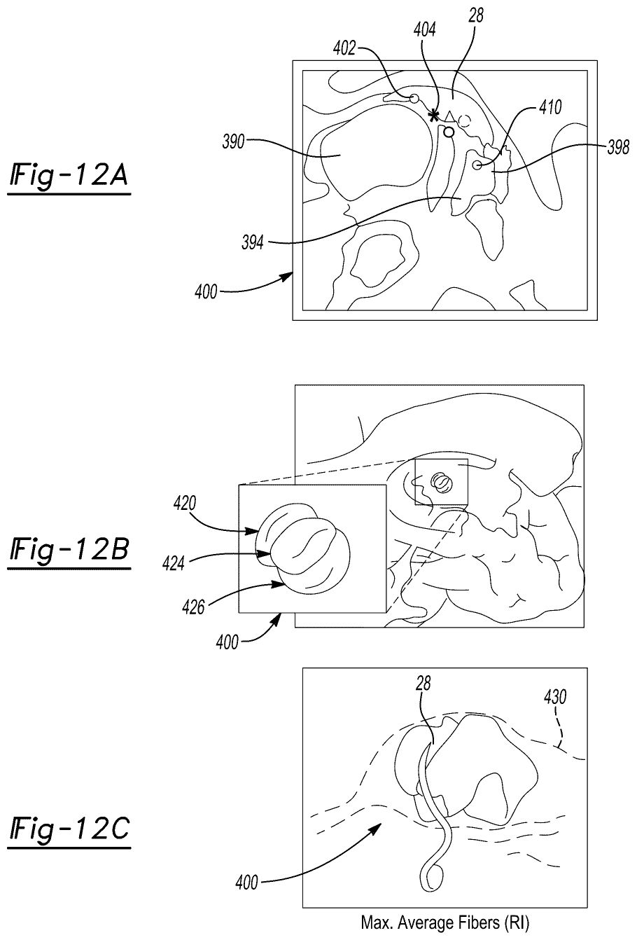

As noted above, the POS 54 may include selected features 62, as discussed above, including the dynamics of response of outcome of a subject, amplitude, and the like. In addition, the selected structures 58 may also be included. As discussed above the selected structures may include those as illustrated in FIG. 1B, such as the thalamus 28, including the ANT 30, and the like. In various embodiments, with reference to FIG. 12A, 12B, and 12C the POS 54 may include specific selected structures 58.

For example, with reference to FIG. 12A specific points which may include three-dimensional position within a model or map, such as an atlas, of a brain may be identified. For example, as illustrated in FIG. 12A, the ANT 30 may be identified as an anatomical structure along with other anatomical structures such as an anterior median thalamus 390, a lateral portion or a lateral nuclei 394, or a ventral interior nucleus 398. Accordingly, the anatomical portions may be identified in the brain atlas that may be illustrated as the atlas 400. Relative to the various anatomical portions may be one or more points that are determined or identified as various points that relate to implant positions for outcomes such as responders, improvers, and no benefit. For example, responder points may be identified as responder points 402 and 404. It may be further understood that a non-responder portion (e.g., position may also be identified such as portion 410. Thus, the points may be identified as positions relative to various anatomical portion in the atlas 400.

The atlas 400 may be displayed on the display 150, as discussed above, but is not required to be so. Further once the atlas 400 is registered to the acquired subject data in block 88 of the process 50, the identified portions and/or points 402, 404, 410 may also be mapped or warped to the subject 20. Selected warping techniques are known to those skilled in the art to register portions within an atlas to an image of the current subject 20. Various warping techniques include age segmenting an atlas and selecting an age appropriate atlas for the current subject. The appropriate atlas is then registered in increasing resolution for at least two steps. A probabilistic system is then used to perform the registration of the atlas to the subject image and warp the features from the atlas, which may include the POS 54 features, to the image of the current subject. Thus, these specific points may be identified and included as selected or possible target points for continued analysis in the process 50 and may be also identified as POS selected structures 58.

Similar to the points or portions identified or illustrated in FIG. 12A, maps of volume of activation may also be made. As discussed above, various volume of activation regions may be identified relative to the anatomical structures, such as those illustrated in FIG. 12A, and/or relative to general regions in the atlas 400. Again, the atlas 400 may include information or be generated based upon a plurality of subjects. Accordingly, VATs, as discussed above, may also be identified and displayed and/or determined relative to the atlas 400. These may relate to outcomes that are no benefit or no response 420, an improvement 424, and/or a responder 426. Each of the volumes may be known and included in the POS 54 as selected structures 58. Again, these may be mapped to the subject 20 after the registration of the POS 54 to the subject in block 88.

Additional structures may include various fibers, such as fiber tracks. The fiber tracks may be identified as structures and include connections between various anatomical structures and may include those as illustrated in FIG. 12C. Again, the atlas 400 may include various anatomical structures such as the ANT 30. Additionally fiber structures, such as a fiber structure 430 may be identified in the atlas 400. The fiber 430 may also be mapped to the subject 20. As discussed above, and is generally known in the art, the acquired subject data 80 may include diffusion data of the subject 20. Thus, fibers may be identified in the subject 20 based upon the diffusion data or appropriate data. This allows the fibers 430 in the selected structures 58 to be mapped to the subject 20 to assist in determining a position and plan for a treatment of the subject, such as placement of the electrode of the implant 34.

Thus, the POS 54 may include the selected features 62 and selected structures 58 as discussed above. These allow for the process 50, as discussed further herein, to be used to assist in determining a therapy of the subject 20 such as a placement of the implant 34, positioning of electrodes on the implant 34, an amplitude or type of therapy, or other appropriate features.

The analysis or determination of a target portion, such as a structure, may be based upon the process 50 as illustrated in FIG. 2. The process 50, as discussed above, includes transforming the subject data to the POS in block 88 and then selecting a type of implant of block 92, selecting a target in block 96, and evaluating a selected target with the POS in block 100. Based upon this various renderings and displays may be made to assist in determining a selected therapy for the subject, that may include an optimal or selected optimal therapy for the subject.

An evaluation of the plurality of selected features or dimensions in block 62 or structures in block 58 may be made in the POS 54 are initially evaluated based on weights selected in block 98. These weights may then be combined to illustrate or determine a sweet spot target position (e.g., structure, point, region, etc.) which may be a selected position or optimal position for a therapy.

The various information, including the selected features 62 may be weighted in block 98 to produce an outcome or an output that is evaluated in block 100 and output in selected manners, such as a visual display thereof, in block 108. Weighting the various features or dimensions from block 62 may be weighted by a user and/or automatically by a system, such as a machine-learning system as discussed further herein. The weight applied to the different features may include those features discussed above and/or any appropriate features included in the POS 54. It is understood that the above-discussed features are exemplary and that other appropriate features may also be defined and included in the POS 54. Further, a user may select various features from the complete features set that are included in the selected feature 62. For example, the user may select to disregard a stability of outcome dimension and/or select a weight for the stability outcome to be zero. Accordingly, the user can identify various features and assign a weight to each one for the evaluation of the selected target with the POS 54.

The evaluation in block 100 may include a summation of the weighted features and an evaluation of the position that may also be referred to as a sweet spot or a sweet spot may be a maximum of the weights for various features. Accordingly, in Equation 1 a multilayer sweet spot may be calculated or determined based upon the maximum value or optimum value of the map represented as LS(x)^{k} Equation 1 is:

$${LS ( x )}^{k} = \frac{1}{L} {\sum }_{i = 1}^{L} {w}_{i}^{k} {S}_{i}^{k} ( x )$$

The LS is a 3D map of probabilities, where the highest probability of being a responders is a local maximum. Therefore, the maxima of the equation are high-dimensional subspace of the optimality space and cannot be explored by subject. One searches for the maximum of the map LS that is computed/represented by Equation 1.

The sweet spot may be a maximum value for the position x of the electrode or implant within a region, such as in the ANT 30 for applying the therapy to the subject. The maximum value has an upper limit of one. In Equation 1, wᵢ^{k} denotes the weight of the i-th outcome map denoted as Sᵢ^{k}. The set of maps include all of those identified above or may be included in the selected features 62 and therefore may be numbered from 1 to L. Thus, the position x may be a voxel or portion of a voxel or pixel of a subject image k. Therefore, the maximum value of Equation 1 may be used to identify a sweet spot based upon the acquired subject data from block 80 and various POS data from block 54. The weights may be altered to maximize or determine a maximum of the Equation 1, as discussed further herein. Thus, the process 50, as discussed above, may adjust the weights in block 112 to an attempt to achieve a maximum for Equation 1.

In addition to the sweet spot identified by Equation 1, a multiplicative combination of the sweet spot value and a weighted target based upon the selected structures, such as the selected structures 58, may be determined by Equation 2. Equation 2 is:

$${ML ( x )}^{k} = \frac{1}{L} {\prod }_{f = 1}^{G} {w}_{f}^{k} {F}_{f}^{k} ( x ) {\sum }_{i = 1}^{L} {w}_{i} {S}_{i}^{k} ( x )$$

The multiplicative combination in Equation 2 allows for an optimization of the term ML(x)^{k}, a term regarding both the sweet spot and of weighting various structures, such as fibers, that may be included or identified in atlas maps and based upon points. In Equation 2, the summation is the summation of Equation 1. The additional multiplicative factor includes the fiber f and the f-th fiber represented as F_{f}^{k} and the related weight thereof w_{f}^{k}. Thus, the maximum value of Equation 2 accounts for both the weighted fiber bundle and the weighted features. Again, the weights may be assigned to determine a maximum of the Equation 2 to determine a target and sweet spot to achieve the selected outcome for the subject 20. As discussed further herein, the weights may be adjusted for Equation 1 alone and/or in Equation 2 in block 112 to achieve a maximum of the selected equation.

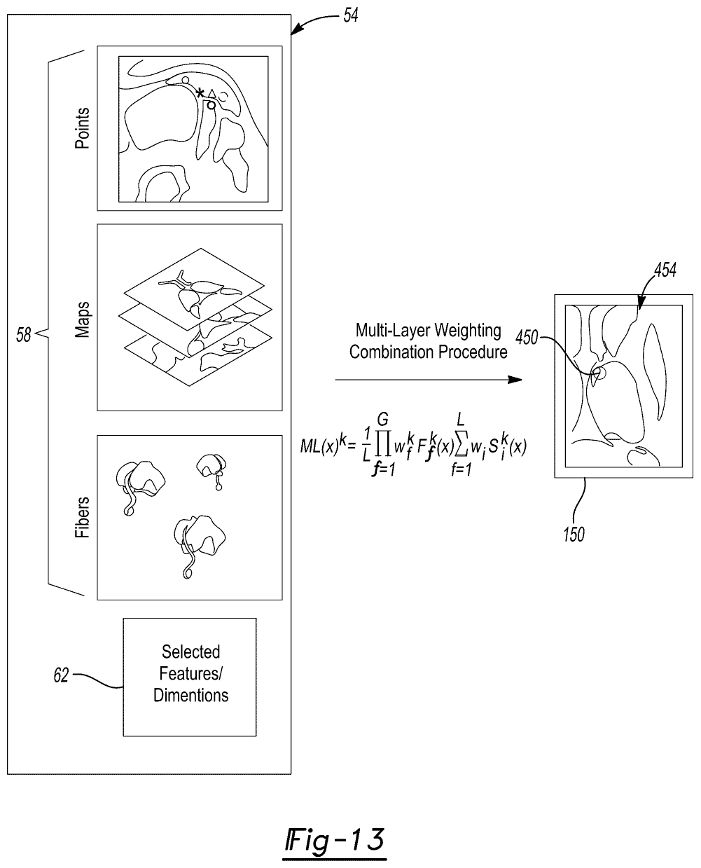

With reference to FIG. 13, a schematic of a visualization including the weights of Equation 2 is illustrated. Various selected structures 58 in the POS 54 may include the points, as illustrated in FIG. 12A, map or atlases as illustrated in FIG. 12B, and fibers as illustrated in FIG. 12C. These structures may be weighted and combined to assist in identifying a target in the subject 20. Further, the selected features and dimensions 62 may also be included and weighted in the determination of a sweet spot target for the subject 20. The POS 54 may be evaluated with the Equation 2 to output a target in the subject 20.

Image data of the subject 454 may be displayed on the display device 150. The subject image data 454 may be a part of the acquired subject data 80, as discussed above. The target 450 may be a sweet spot target that is identified based upon the POS 54 and the subject data as determined in the evaluation 100. Thus, the target 450 may be a target after selected weighting of the features and structures, such as the fibers of the atlas, to determine the target 450. The weights may be adjusted to adjust the target 450 to determine a maximization of the Equation 1 or Equation 2 to determine one or more possible targets. A user may select the final target based upon the maximum of a map generated by the Equations 1 or 2 and/or other selected determinations. Nevertheless, the target may be displayed on the subject data 454 and displayed for visualization by the user and/or for planning and/or for placement of the implant 34.

Various visualization may be used to determine a position for the implant 34 may include those discussed above, such as the maps as illustrated in FIG. 12B. These visualizations that may be displayed on the display device 150 allow a user to view atlases that may be registered to the subject, as discussed above. Further, as illustrated in FIG. 13, a target may be identified and displayed based upon a selected weighted input in the POS 54. The display 150 may be included in a selected system that may be used by a user 480 at any appropriate time, such as to plan a procedure for placing the implant 34 into the subject, which may be a new subject. The implant 34 may be positioned in the subject at a selected time, such as during an operative time. Planning of positioning of the implant may be performed prior to the operative procedure and/or at any appropriate time. The display 150 may display visualizations, such as a target location 450 superimposed on the image 454, as illustrated in FIG. 14.

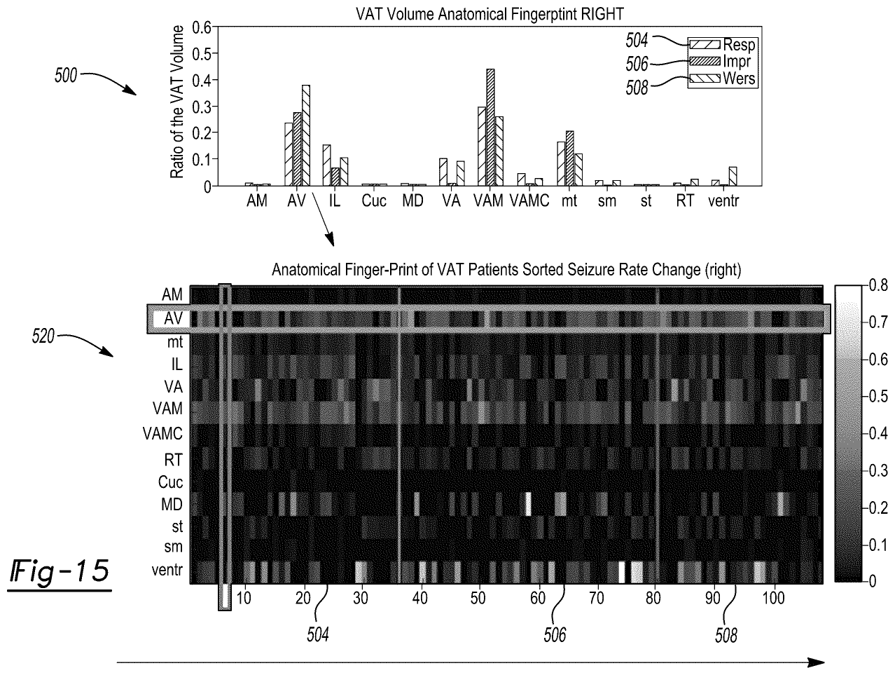

Additional and/or alternative visualizations may also be provided for the user to assist in evaluating a selected therapy to the subject 20. For example, a fingerprint as a graph may be provided to illustrate a number of steady outcome that achieve an outcome based upon a selected volume of activation. For example, as illustrated in FIG. 15, a graph 500 may illustrate on an x-axis, a plurality of anatomical regions, or other structures that are included in and or modulated relative to (e.g., with and/or due to) a VAT. A y-axis may illustrate a percentage of that portion that is activated in a subject. According to various embodiments three type of outcomes may be included including a responder outcome 504, an improver outcome 506, and a no-benefit outcome 508. The graph 500 may provide a fingerprint or an illustration of the results based upon a ratio of the selected area that is activated as a part of the volume of activation based upon a placement and initiation of therapy. The graph 500 may be based on a plurality of subjects and illustrate the various portion that are within a volume of activation that achieve a selected result. For example, an anterior ventral nucleus (AV) may be illustrated to be included in a volume of activation tissue. The three types of outcomes may be graphed based upon the volume of activated tissue at the selected region. It is understood that any appropriate number of regions or structures may be included in the fingerprint graph 500.

For a plurality of subjects, a representation may also be illustrated relative to a plurality of subjects. With reference to graph 520, a plurality of subjects may be included in a normalized grouping where each column represents a single subject and each row represents a single structure within a VAT. The subjects may be grouped similarly to the grouping graph 500 including a responder grouping 504, an improver grouping 506, and a no-benefit grouping 508. The groupings may be illustrated in a selected manner, such as in a gray scale, heat map, or the like, that illustrates a volume of activation of various areas related to the selected patient. Therefore, each row of the heat map 520 may relate to a single structure that is within and/or effected by the volume of activation. For example, the row identified as AV may illustrate a volume of activation that is generally lighter for subjects that are responders as opposed to those that have no benefit.

Thus, a user may view the fingerprint graph 500 and/or the heat map graph 520 to assist and understand or selecting a volume to be activated and a possible or probable outcome based upon the volume of activation. This visualization may be displayed on the display 150 and assist the user in identifying a selected outcome, such as weighting selected portions of the POS 54, to assist in defining or selecting an implant and selected therapy therefore.

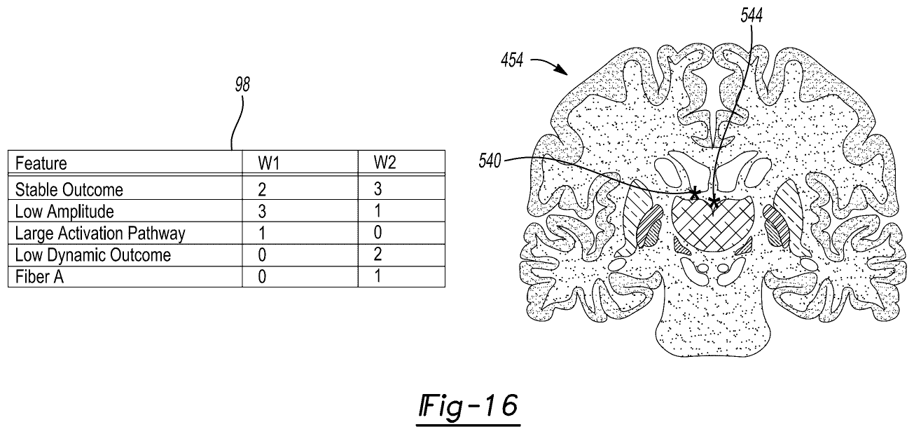

The process 50, therefore, may be used to identify a target location for placing a selected implant for providing therapy to the subject. Returning reference to FIG. 2, the adjustment sub-process 127 allows for adjustment of one or more weights for various features or structure. With additional reference to FIG. 16, for example, weights may be selected for various features and/or structures. As exemplary illustrated in FIG. 16, the features include stable outcome, low amplitude, large-activation pathway, and low-dynamic outcome. These various features are discussed above and may be included with other features, if selected. Further, exemplary fiber A is selected or weighted as a structure. Fiber A may be any appropriate fiber portion or structure that may be identified in a selected atlas.

With reference to FIG. 16, the weighting block 98 includes a first weight that may include weights as illustrated at W1. The weights may be evaluated in block 100 and rendering an evaluation may be optionally made in block 104. Based upon the evaluation and rendering, a display may optionally be made, as illustrated in FIG. 16. For example, the sweet spot position based upon the selected structures 58 and selected features 62 as weighted in block 98 as W1, may be illustrated as a target location 540. The target location may be illustrated superimposed on the subject image 454, as discussed above. Based upon various selections, such as relative to a threshold, weights may be adjusted. A threshold may include a selected location or distance from a surface of an anatomical structure, such as the thalamus 28, a position relative to other anatomical structures, such as a ventricle, or other features, such as a volume of activation, may be used to determine an adjustment of the weights in block 112.

The weights may be adjusted in block 112 to generate the weights W2, as illustrated in FIG. 16. According to various techniques, a machine-learning system may be implemented to adjust the weights to optimize the maps generated with the Equation 1 and Equation 2, as discussed above. Also, a manual adjustment of the weights may be made to generate the W2 weights in block 98, as illustrated in FIG. 16. In an automatic or machine-learning process, the machine-learning process may also adjust the weights to attempt to reach a target location that is within a selected threshold, including within the limitations noted above. Limiting values for thresholds may relate to the probability of adverse effects. Further limitations would origin in the desired time evolution of the outcomes, knowing that some less volatile outcome time course contributes to better outcomes. The selected weights, such as the selected W2 weights, may also then be used to evaluate in block 100 and render and display in blocks 104, 108. The displayed target based upon the W2 weight may be target 544, which may also be illustrated relative to the subject image 454.

Thus, the adjustments of the weights in block 112, as illustrated in FIG. 16, may include a different weighting of various features or structures to achieve different target locations relative to the subject image 454. The user may adjust these weights based upon various analysis, such as a selection by the user for placement of an implant, the experience of the user, or the like. Further a machine-learning system may adjust the weights based upon an initial input in an attempt to achieve a maximization of the probability of best therapeutic outcome as generated by the Equation 1 and 2, as discussed above.

The machine-learning system may be trained on selected data to achieve the outcome maximization based upon the selected inputs or selected weights. Based upon the initial output, the machine-learning system may be trained to adjust the weights, such as W2 relative to W1, to achieve a sweet spot that maximizes probability of best outcomes or other selected values and/or is within a selected threshold location of the target.

Thus, the process 50 to be executed substantially entirely by a processor system that is executing instructions, such as a processor system or processor module 550 that may be incorporated with a selected user system, such as a computer system 554. The instructions executed by the processor module 550 may be stored in any appropriate location, such as in a memory system 558. The machine-learning system may be trained and the trained system may be stored in the memory 558. The memory system 558 may also allow image data or data of the subject 20 to be accessed by the processor 550 to evaluate based upon the input subject data and the POS 54, as discussed above. The weights may then be adjusted, such as substantially automatically with the machine-learning system, to achieve a selected outcome that may be the sweet spot. Additionally, and/or alternatively, the user 480 may adjust the weights. In various embodiments, the user 480 may provide an initial weight and a machine-learning system may assist in evaluating or selecting additional weights for further analysis.

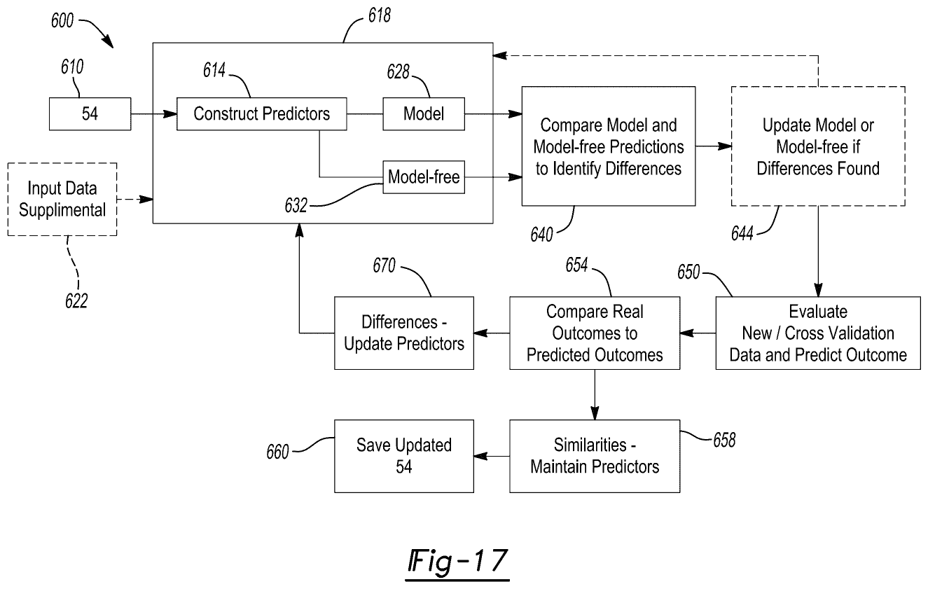

Turning reference to FIG. 17, a process 600 may be used either with the process 50, discussed above, such as after the end block 130 and/or separately to generate the POS 54 for the process 50. The process 600 may be executed by a processor system executing instructions based upon the process of the flow chart 600 and may include an input of the POS 54 in block 610.

As discussed above, the POS 54 may include a plurality of structures and/or features that may be used to evaluate a subject data to determine a target for a therapy. The POS 54 may be based upon a plurality of inputs, such as outcomes and the like determined based upon an evaluation of a plurality of subjects. Further, the structures may be based upon an atlas that is used to identify various structures or portions within a group of subjects.

Accordingly, the POS 54 may be input or provided to the system 600 to construct predictors in block 614. The constructed predictors may be in a predictor and model block or sub-block 618. Further additional data, if selected, may also be provided as supplemental to the POS data in block 622. Supplemental data 622 may include new or additional features and/or specific applications that may be included in the construction of the predictor 614.

The construction of the predictor 614 may include predictors based upon possible outcomes. As discussed above, possible outcomes may include an improver outcome, a responder outcome, or a no-benefit outcome. Other appropriate outcomes may also be identified and the predictors may be used to assist in predicting an outcome based upon the input data or information, including the POS 54. Accordingly, predictors may be generated and identified based upon various modeling schemes such as model-based predictors 628. Non-modeling schemes may also be used to generate predictors in model free predictor methods 632. The two methods, the model method 628 and the model free method 632 may be used to generate the predictors or predict possible outcomes based upon the input data.

The model-based predictors 628 may include various techniques such as multivariant or multinomial regression methods. Various methods may include linear and non-linear methods, such as one or more mixed effect models. These model-based methods generally use regression techniques to estimate predictors and evaluate various features such as placement of an implant, therapy amplitude, and the like. Each of the model inputs may be a specific model method or value that is associated with a specific coefficient. These various regression methods may then be minimized to attempt to optimize or reach a maximum value based upon the modeling technique. Various regression techniques include LASSO regression that may assist in a selection of both variables and values thereof to model a system, including the outcomes discussed above.

The model-free method 632 may include various machine-learning systems, including deep-learning systems. Model-free systems 632 include generating a machine-learning system that is taught based upon various inputs to achieve selected outputs. The model-free system 632 need not include specific modeling calculations and/or specific coefficients for specific features.

In the process 600, a comparison of model and model-free prediction to identify differences may then be made in block 640. Accordingly, the model prediction 628 and the model-free prediction 632 may be input and compared in block 640. Based upon a comparison, an update of the model or the model-free systems may be made if differences are found in block 644. It is understood that an update of the model and model-free system is not required at this point, but may be done if the comparison finds differences that are of a selected degree or threshold. The models may be updated before evaluating further subject data based upon the models in block 644.

Regardless of whether the model 628 or the model free 632 is updated in block 644, an evaluation of new or cross validation data is made in block 650. The new or cross validation data may include data separate from that used to generate the POS 610 and/or the supplemental input data 622. Thus, the models may be used to generate predictions. These predictions may then be compared to real outcomes in block 654. Thus, the new or cross validation data may be evaluated with the model 628, 632 in block 650 in a comparison of the predicted outcomes to the actual or real outcomes of block 654 may be made.

If there are only similarities or at least threshold similarities between the comparison of the actual outcomes and the predicted outcomes a similarities path 658 may be followed to save the POS and/or updated POS in block 660. Thus, the POS 54 may be saved and used for the process 50 to predict or select a target for a subject.

If differences are determined in the comparison block 654, a differences path 670 may be followed to the construct predictors subroutine 618. If the differences are determined to be greater than a selected threshold, the differences may be used to assist in reevaluating or reconstructing the predictors including the model 628 or the model-free 632. The threshold for initiating the updates may be computed with statistical tests for two groups, for example a significant t-test (p<0.05) for the two predictor groups may initiate an update. A further test may include the Wilcoxon rank sum test that estimates if the two groups are different.

For example, if differences are found between the predicted outcomes and the actual outcomes the model may be evaluated based upon a specific input coefficient and/or additional inputs. In various embodiments, if the model is based on a DBS position and amplitude and differences between the prediction based upon the model 628 and the actual outcomes are found additional features may be evaluated or added to the POS 54 and/or the values or weights thereof may be augmented in the POS 54.

In the model-free system 632, the machine-learning system may be trained with the additional data including the cross validation of new data in block 650. The additional training data may be used to train the machine-learning system to provide greater similarity between the predictions and the actual outcomes to minimize differences in the comparison block 654.

Therefore, the process 600 allows for an evaluation of the POS 54 and/updating of the POS 54. Thus, the POS 54 used in the method 50 to evaluate and determine a target for the subject may be constantly updated based upon additional data of various subjects. For example, a current subject may be treated with a selected treatment, such as with a positioning of a DBS and a selected therapy type. The actual outcome of the current subject may then be used as new or cross-validation in block 650. The comparison of the prediction to the actual outcome in block 654 may allow for an updating or validation of the POS 54. Thus, the POS 54 may be maintained for providing selected or optimal outcomes for future subjects.

As discussed above, therefore, the subject 20 may have the implant 34 positioned therein to assist in providing a therapy to the subject 20. Returning reference to FIG. 14, the implant 34 may be positioned within the subject 20 by the user 480. The processing system 554 may assist in evaluating the planning for placement of the implant 34. Further, the processing system 554 and/or various additional systems may also be used to assist in performing the procedure on the subject 20. In various embodiments, including those discussed above, the implant 34 may be positioned in the subject 20 in an appropriate manner. In various embodiments, for example, the implant 34 may be guided into the subject 20 with the selected navigation system, such as a surgical navigation system including those sold by Medtronic Navigation, Inc. such as the Stealth Station® surgical navigation system.

Briefly, in surgical navigation, the subject may be tracked with a selected tracking device, such as a subject tracking device 700. The subject tracking device may be tracked with an appropriate tracking system such as an electromagnetic tracking system 704 and/or an optical tracking system 708. It is understood that other appropriate tracking systems may be used and the EM 704 and optical 708 tracking systems are merely exemplary.

The tracking systems may track the position of the patient 700 and maintain a registration of a patient space to find relative to the patient tracking device 700 to an image displayed on the display 150, such as the subject image 454. A device representation, such as a graphical representation thereof, 710 may be displayed on the display device 150 relative to the subject image 454 based upon a tracked location of the device 34. A device tracking device 714 may be tracked with the tracking system 704, 708, as is understood by one skilled in the art. It is also understood by one skilled in the art that the tracked position and navigated position of the device 34 may be performed based upon a registration of the image or image space 454 to the subject or subject space of the subject 20. As discussed above various registrations may also occur between the subject image 454 and/or additional image, such as structures in the POS 54.

The positioning of the device 34 may be performed in a selected suite, such as a surgical suite 730. The surgical suite may include selected structures or portions such as a patient support 734 and an imaging system 738. The imaging system 738 may be used to generate or acquire image data of the subject 20, according to various embodiments. The imaging system 738 may also be tracked with an imaging system tracker 742, as is generally understood by one skilled in the art. Various imaging systems may include an O-arm® imaging system, sold by Medtronic, Inc. Thus, the implant 34 may be positioned within the subject 20 based upon the plan or target location, such as identified with the process 50, as discussed above.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms and that neither should be construed to limit the scope of the disclosure. In some example embodiments, well-known processes, well-known device structures, and well-known technologies are not described in detail.

Instructions may be executed by a processor and may include may include software, firmware, and/or microcode, and may refer to programs, routines, functions, classes, data structures, and/or objects. The term shared processor circuit encompasses a single processor circuit that executes some or all code from multiple modules. The term group processor circuit encompasses a processor circuit that, in combination with additional processor circuits, executes some or all code from one or more modules. References to multiple processor circuits encompass multiple processor circuits on discrete dies, multiple processor circuits on a single die, multiple cores of a single processor circuit, multiple threads of a single processor circuit, or a combination of the above. The term shared memory circuit encompasses a single memory circuit that stores some or all code from multiple modules. The term group memory circuit encompasses a memory circuit that, in combination with additional memories, stores some or all code from one or more modules.

The apparatuses and methods described in this application may be partially or fully implemented by a special purpose computer created by configuring a general purpose computer to execute one or more particular functions embodied in computer programs. The computer programs include processor-executable instructions that are stored on at least one non-transitory, tangible computer-readable medium. The computer programs may also include or rely on stored data. The computer programs may include a basic input/output system (BIOS) that interacts with hardware of the special purpose computer, device drivers that interact with particular devices of the special purpose computer, one or more operating systems, user applications, background services and applications, etc.

The computer programs may include: (i) assembly code; (ii) object code generated from source code by a compiler; (iii) source code for execution by an interpreter; (iv) source code for compilation and execution by a just-in-time compiler, (v) descriptive text for parsing, such as HTML (hypertext markup language) or XML (extensible markup language), etc. As examples only, source code may be written in C, C++, C #, Objective-C, Haskell, Go, SQL, Lisp, Java®, ASP, Perl, Javascript®, HTML5, Ada, ASP (active server pages), Perl, Scala, Erlang, Ruby, Flash®, Visual Basic®, Lua, or Python®.

Communications may include wireless communications described in the present disclosure can be conducted in full or partial compliance with IEEE standard 802.11-2012, IEEE standard 802.16-2009, and/or IEEE standard 802.20-2008. In various implementations, IEEE 802.11-2012 may be supplemented by draft IEEE standard 802.11ac, draft IEEE standard 802.11ad, and/or draft IEEE standard 802.11ah.

A processor or module or ‘controller’ may be replaced with the term ‘circuit.’ The term ‘module’ may refer to, be part of, or include: an Application Specific Integrated Circuit (ASIC); a digital, analog, or mixed analog/digital discrete circuit; a digital, analog, or mixed analog/digital integrated circuit; a combinational logic circuit; a field programmable gate array (FPGA); a processor circuit (shared, dedicated, or group) that executes code; a memory circuit (shared, dedicated, or group) that stores code executed by the processor circuit; other suitable hardware components that provide the described functionality; or a combination of some or all of the above, such as in a system-on-chip.

The foregoing description of the embodiments has been provided for purposes of illustration and description. It is not intended to be exhaustive or to limit the invention. Individual elements or features of a particular embodiment are generally not limited to that particular embodiment, but, where applicable, are interchangeable and can be used in a selected embodiment, even if not specifically shown or described. The same may also be varied in many ways. Such variations are not to be regarded as a departure from the invention, and all such modifications are intended to be included within the scope of the invention.

1-83. (canceled)

84. A method of generating an optimality data for planning a procedure with a current subject data to plan a position for placement of an implant in a current subject, the method comprising:
accessing an optimality data including at least one feature data regarding a specific therapy and at least one structure data regarding a specific therapy, wherein the feature data and the structure data includes data regarding possible positions of the implant, possible therapies with the implant, and possible outcomes related to the possible positions and therapies;
generating predictors based on the accessed optimality data;
evaluating a validation subject data to predict an outcome based on the generated predictors;
determining a similarity between the predicted outcome and a real outcome;
if the similarity is below a selected threshold, update the accessed optimality data to generate an updated optimality data; and
saving the updated optimality data when generated.

85. The method of claim 84, wherein generating predictors based on the accessed optimality data includes evaluating a model to determine the predictors;
wherein the model includes a regression analysis.

86. The method of claim 84, wherein generating predictors based on the accessed optimality data includes evaluating the optimality data with a model-free system to determine the predictors;
wherein the model-free system includes a machine learning process.

87. The method of claim 86, wherein the machine learning process is a deep learning process.

88. The method of claim 84, wherein generating the predictors based on the accessed optimality data includes generating the predictors with at least a model method and a model-free method; and
further comprising comparing model predictors and model-free predictors.

89. The method of claim 88, further comprising:
updating at least one of the model predictors or model-free predictors based on the comparison.

90. The method of claim 84, further comprising:
selecting at least a first classification and a second classification for the real outcome;
wherein the generated predictors are operable to predict either the first classification or the second classification as the outcome.

91. The method of claim 90, further comprising:
accessing the validation subject data including a determination of the real outcome based on the validation subject data;
wherein the determining the similarity between the predicted outcome and the real outcome includes determining whether the real outcome is the same classification as the predicted outcome.

92. The method of claim 84, further comprising:
providing access to a planning system of the saved updated optimality data.

93. The method of claim 84, further comprising:
performing a selected treatment on the current subject based upon the updated optimality data.

94. The method of claim 93, wherein the selected treatment is a positioning of a DBS implant relative to the current subject.

95. The method of claim 94, further comprising:
evaluating the actual outcome of the selected treatment of the current subject and updating the validation subject data based upon the selected treatment.

96. The method of claim 95, further comprising:
using the updated validation subject data for a future subject.

97. A system operable to generate an optimality data for planning a procedure with a current subject data to plan a position for placement of an implant in a current subject, comprising:
a processor module configured to execute instructions to:
access an optimality data including at least one feature data regarding a specific therapy and at least one structure data regarding a specific therapy, wherein the feature data and the structure data includes data regarding possible positions of the implant, possible therapies with the implant, and possible outcomes related to the possible positions and therapies;
determine predictors based on the accessed optimality data;
evaluate a validation subject data to predict an outcome based on the generated predictors;
determine a similarity between the predicted outcome and a real outcome;
determine if the similarity is below a selected threshold to update the accessed optimality data to generate an updated optimality data; and
an output system to output the updated optimality data when generated for recall.

98. The system of claim 97, further comprising:
a memory system configured to save the output updated optimality data.

99. The system of claim 97, further comprising:
a user input system to input the real outcome of the validation subject data.

100. The system of claim 97, wherein the processor module is further configured to execute instructions to generate predictors based on the accessed optimality data by evaluating a model to determine the predictors;
wherein the model includes a regression analysis.

101. The system of claim 97, wherein the processor module is further configured to execute instructions to generate predictors based on the accessed optimality data by evaluating a model-free system to determine the predictors;
wherein the model-free system includes a machine learning process.

102. The system of claim 97, wherein the processor module is further configured to execute instructions to compare model predictors and model-free predictors; and
update at least one of the model predictors or model-free predictors based on the comparison.

103. The system of claim 97, wherein the output system is configured to further provide an output position of a target in the current subject for positioning of the implant.
